(19) United States
(12) Patent Application Publication
(10) Pub. No.: US 2024/0309396 A1
(43) Pub. Date: Sep. 19, 2024

(54) COMPOSITIONS AND METHODS FOR IMPROVED GENE EDITING

(71) Applicant: UNIVERSITY OF MASSACHUSETTS, Boston, MA (US)

(72) Inventors: Jonathan Kenneth Watts, Worcester, MA (US); Erik Joseph Sontheimer, Auburndale, MA (US); Craig Cameron Mello, Barrington, RI (US); Zexiang Chen, Worcester, MA (US); Krishna Sumanth Ghanta, Grafton, MA (US)

(21) Appl. No.: 18/424,165

(22) Filed: Jan. 26, 2024

Related U.S. Application Data
(63) Continuation of application No. PCT/US2022/074467, filed on Aug. 3, 2022.
(60) Provisional application No. 63/229,213, filed on Aug. 4, 2021.

(51) Int. Cl. C12N 15/85; C12N 15/11

(52) U.S. Cl. CPC C12N 15/85; C12N 15/11; C12N 2310/315; C12N 2310/3519

(57) ABSTRACT

The disclosure provides methods and compositions for gene editing. In particular. the disclosure relates to compositions and methods of making and using modified nucleic acid donor templates for highly efficient and precise gene editing, such as gen editing in immune cells.

### RELATED APPLICATIONS

This application claims the benefit of U.S. Provisional Application No. 63/229,213, filed Aug. 4, 2021, the content of which is incorporated by reference in its entirety for all purposes.

### STATEMENT OF FEDERALLY SPONSORED RESEARCH

This disclosure was made with government support under Grant No. TR002668 awarded by the National Institutes of Health. The Government has certain rights in this disclosure.

### SEQUENCE LISTING

The instant application contains a Sequence Listing which has been submitted electronically in XML format and is hereby incorporated by reference in its entirety. Said XML file, created on Jan. 26, 2024, is named 749691_UM9-266PCCON_ST26.xml and is 61,537 bytes in size.

### FIELD OF THE DISCLOSURE

The instant disclosure provides novel methods and compositions for gene editing. In particular, the disclosure relates to compositions and methods of making and using modified nucleic acid donor templates for highly efficient and precise gene editing, such as in immune cells.

### BACKGROUND

In recent years, several approaches have been developed for genome editing in eukaryotic systems including mammalian cells, such as somatic or germline cells, zygotes or embryos, plants, rodents, worms, insects, and many other organisms. In all cases a nuclease (clustered regularly interspaced short palindromic repeats [CRISPR]-associated Cas9, Cas12a/Cpf1, transcription activator like effector nuclease [TALEN], zinc finger nuclease [ZFN], etc.) is used to generate a targeted DNA lesion, which is resolved as a precise or imprecise edit by the cell's DNA repair machinery. Both precise and imprecise editing approaches have found applications in gene therapy, agriculture, development of research tools, and elsewhere. Precise genome editing, in which the targeted sequence is re-written in a user-defined fashion requires the introduction of a donor template that the cell machinery can use in gene editing mechanisms such as homology-directed DNA repair (HDR) or homology independent targeted integration (HITI). Routine use of genome editing methodology is currently hampered by the low frequency of precision gene editing in many model organisms and cell lines. This is particularly true of longer donor templates. Accordingly, there exists a need in the art for nucleic acid donor templates with improved gene editing activity.

### SUMMARY

The present disclosure provides compositions and methods for improved gene editing, e.g., via homology-directed repair (HDR) or homology-independent targeted integration (HITI). For example, the compositions and methods of the disclosure may improve precision gene editing of a diverse array of donor nucleic acid acids templates, including single-stranded and double-stranded DNA templates of various sizes and lengths. Further, the compositions and methods of the disclosure can be used in various host organisms and cells, including, but not limited to, human and other mammalian subjects. The methods and compositions are useful in a variety of gene editing and genome engineering strategies and contexts. For example, in some embodiments, the compositions and methods are useful for repairing mutations (e.g., heterozygous mutations) that are widely found in patients having certain diseases (e.g., monogenic recessive diseases). In other embodiments, the compositions and methods are useful for introducing an exogenous gene of interest (GOI) to the genome of a host cells. It will be appreciated, however, that the compositions and methods of the disclosure are not limited to editing a specific gene or mutation.

In certain aspects, the disclosure provides an isolated nucleic acid donor sequence comprising a 5′ end and a 3′ end, wherein a single-stranded nucleic acid (ssNA) moiety is attached at the nucleic acid donor sequence 5′ end, and wherein the ssNA comprises at least three phosphorothioate internucleotide linkages.

In another aspect, the disclosure an isolated nucleic acid donor sequence comprising a 5′ end and a 3′ end, wherein a single-stranded RNA (ssNA) moiety is attached at the nucleic acid donor sequence 5′ end, and wherein the ssNA comprises an immunosuppressive sequence.

In certain embodiments, the nucleic acid donor sequence comprises a region having portions of nucleic acid homology to a target sequence.

In certain embodiments, the nucleic acid donor sequence is introduced into a target sequence by a homology-independent integration mechanism.

In certain embodiments, the homology-independent integration mechanism comprises engineering a cleavage site sequence into the nucleic acid donor, wherein the cleavage site sequence is also present in the target sequence.

In certain embodiments, the ssNA moiety comprises a single stranded RNA (ssRNA).

In certain embodiments, the ssNA or the ssRNA comprises a sequence selected from the group consisting of:
(mG)(mG)(mA)(mA)(mG)(mG)(mG)(mC)(mC)(mG)(mA)(mG)(mC)(mG)(mC);
(mG) #(mG) #(mA) #(mA)(mG)(mG)(mG)(mC)(mC)(mG)(mA)(mG)(mC)(mG)(mC);
(mG) #(mG) #(mA) #(mA) #(mG) #(mG) #(mG) #(mC) #(mC)(mG)(mA)(mG)(mC)(mG)(m C);
(mG) #(mG) #(mA) #(mA) #(mG) #(mG) #(mG) #(mC) #(mC) #(mG) #(mA) #(mG) #(mC) #(m G) #(mC) #;
(dG)(dG)(dA)(dA)(dG)(dG)(dG)(dC)(dC)(dG)(dA)(dG)(dC)(dG)(dC);
(dG) #(dG) #(dA) #(dA)(dG)(dG)(dG)(dC)(dC)(dG)(dA)(dG)(dC)(dG)(dC);
(dG) #(dG) #(dA) #(dA) #(dG) #(dG) #(dG) #(dC) #(dC)(dG)(dA)(dG)(dC)(dG)(dC);
(dG) #(dG) #(dA) #(dA) #(dG) #(dG) #(dG) #(dC) #(dC) #(dG) #(dA) #(dG) #(dC) #(dG) #(dC) #;
(mA)(mA)(mG)(mA)(mA)(mG)(mA)(mA)(mG)(mA)(mA)(mG)(mA)(mA)(mG);
(mU)(mU)(mC)(mU)(mU)(mC)(mU)(mU)(mC)(mU)(mU)(mC)(mU)(mU)(mC);
(dT) #(dT) #(dA) #(dG) #(dG) #(dG) #(dT) #(dT) #(dA) #(dG) #(dG) #(dG) #(dT) #(dT) #(dA) #(dG) #(dG) #(dG) #(dT) #(dT) #(dA) #(dG) #(dG) #(dG) #; or
(mU) #(mU) #(mA) #(mG) #(mG) #(mG) #(mU) #(mU) #(mA) #(mG) #(mG) #(mG) #(mU) #(mU) #(mA) #(mG) #(mG) #(mG) #(mU) #(mU) #(mA) #(mG) #(mG) #(mG) #,
wherein “m” corresponds to a nucleotide with a 2′-OMe modification; “d” corresponds to a nucleotide with a 2′H modification; and “#” corresponds to a phosphorothioate internucleotide linkage.

In certain embodiments, the ssNA moiety is attached to the 5′ end of the nucleic acid donor sequence with a linker.

In certain embodiments, the linker is selected from the group consisting of aminoethoxyethoxyacetate (AEEA), aminohexanoic acid, oligoglycine, ethylene glycol, polyethylene glycol (PEG), amino C6, and amino C12.

In certain embodiments, the linker comprises triethylene glycol or tetraethylene glycol.

In certain embodiments, the ssNA further comprises one or more of ethylene glycol, polyethylene glycol (PEG), a polyamine having at least two amino groups, and an alkanediol attached to the 5′ end of the ssNA.

In certain embodiments, the ssNA comprises one or more modified nucleotides.

In certain embodiments, the one or more modified nucleotides are selected from the group consisting of a 2′-O-alkyl modified nucleotide, a 2′-fluoro modified nucleotide, a nucleotide comprising a 5′-phosphorothioate group, a 2′-deoxy-modified nucleotide, a locked nucleic acid (LNA), a bridged nucleotide, a constrained nucleotide, a bicyclic nucleotide, an abasic nucleotide, a 2′-amino-modified nucleotide, a 2′-alkyl-modified nucleotide, a morpholino nucleotide, a phosphoramidate, a peptide nucleic acid, and a non-natural base comprising nucleotide.

In certain embodiments, the one or more modified nucleotides are 2′-O-methyl (2′-OMe) modified nucleotides.

In certain embodiments, the ssNA comprises one or more modified internucleotide linkages.

In certain embodiments, the modified internucleotide linkage comprises a phosphorothioate internucleotide linkage.

In certain embodiments, the modified internucleotide linkage comprises a modified internucleotide linkage of Formula I:

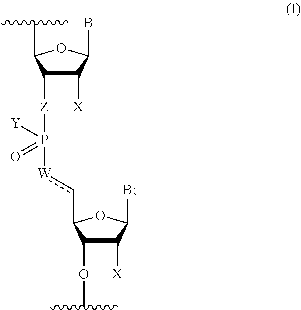

wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O, OH, OR, NH, NH₂, S—, and SH;
Z is selected from the group consisting of O and CH₂;
R is a protecting group; and
[symbol] is an optional double bond.

In certain embodiments, the ssNA is about 1 base in length to about 50 bases in length.

In certain embodiments, the ssNA is about 8 bases in length to about 30 bases in length.

In certain embodiments, the ssNA comprises 3, 4, 5, 6, 7, 8, 9, 10, 11, 12, 13, 14, 15, 16, 17, 18, 19, 20, 21, 22, 23, or 24 phosphorothioate internucleotide linkages.

In certain embodiments, every nucleotide in the ssNA comprises a phosphorothioate internucleotide linkages.

In certain embodiments, every nucleotide in the ssNA comprises a 2′-OMe modified nucleotide.

In certain embodiments, every nucleotide in the ssNA comprises a 2′-deoxy-modified nucleotide.

In certain embodiments, the immunosuppressive sequence comprises (UUAGGG)ₙ or (TTAGGG)ₙ, wherein n is an integer from 1 to 5.

In certain embodiments, administration of the nucleic acid donor sequence comprising the immunosuppressive sequence to a subject elicits an inflammatory response in the subject that is at least 2-fold, at least 5-fold, at least 10-fold, or at least 50-fold lower than a nucleic acid donor sequence that does not comprise the immunosuppressive sequence.

In certain embodiments, the immunosuppressive sequence inhibits production of one or more proinflammatory cytokines.

In certain embodiments, the immunosuppressive sequence inhibits an inflammatory response induced by the toll-like receptor (TLR) pathway.

In certain embodiments, the ssNA binds to a terminal adaptor ligand.

In certain embodiments, the ssNA binds the terminal adaptor ligand through nucleic acid base-pairing interactions.

In certain embodiments, the terminal adaptor ligand comprises a peptide nucleic acid (PNA), a ssRNA, or a ssDNA.

In certain embodiments, the terminal adaptor ligand is attached to a terminal adaptor ligand moiety that confers one or more functionalities to the nucleic acid donor sequence.

In certain embodiments, the one or more functionalities is selected from the group consisting of tissue targeting, PK-modification, and nuclear localization.

In certain embodiments, the terminal adaptor ligand moiety is a peptide, a carbohydrate, a lipid, a steroid, or a small molecule.

In certain embodiments, the terminal adaptor ligand moiety is a nuclear localization signal (NLS).

In certain embodiments, the NLS comprises PKKKRK.

In certain embodiments, the terminal adaptor ligand is attached to the terminal adaptor ligand moiety through a linker.

In certain embodiments, the linker is selected from the group consisting of aminoethoxyethoxyacetate (AEEA), aminohexanoic acid, oligoglycine, ethylene glycol, PEG, amino C6, and amino C12.

In certain embodiments, the nucleic acid donor sequence is double-stranded.

In certain embodiments, the nucleic acid donor sequence is single-stranded.

In certain embodiments, the single stranded nucleic acid is a single stranded donor oligonucleotide (ssODN).

In certain embodiments, nucleic acid donor sequence comprises portions of nucleic acid homology that are about 20 bases in length to about 1,000 bases in length.

In certain embodiments, the nucleic acid donor sequence comprises one or

more phosphorothioate internucleotide linkages at the 5′ end.

In certain embodiments, the nucleic acid donor sequence comprises 1, 2, 3, 4, 5, 6, 7, 8, 9, 10, 11, 12, 13, 14, 15, 16, 17, 18, 19, or 20 phosphorothioate internucleotide linkages at the 5′ end.

In certain embodiments, the phosphorothioate internucleotide linkages are consecutive from the 5′ end.

In certain embodiments, the phosphorothioate internucleotide linkages are alternating from the 5′ end.

In certain embodiments, the nucleic acid donor sequence comprising the ssNA enhances donor genome integration relative to a nucleic acid donor sequence lacking the ssNA.

In certain embodiments, donor genome integration is enhanced about 2-fold or greater.

In certain embodiments, the nucleic acid donor sequence enhances donor genome integration in an immune cell or a hematopoietic stem or progenitor cell (HSPC) relative to a nucleic acid donor sequence lacking the ssNA.

In certain embodiments, donor genome integration is enhanced about 2-fold, about 3-fold, about 4-fold, about 5-fold, about 6-fold, about 7-fold, or greater.

In certain embodiments, the immune cell is a primary immune cell.

In certain embodiments, the primary immune cell is a T cell, a NK cell, or a B cell.

In one aspect, the disclosure provides an isolated nucleic acid donor sequence comprising a 5′ end and a 3′ end, wherein the nucleic acid donor sequence comprises one or more phosphorothioate internucleotide linkages at the 5′ end.

In certain embodiments, the nucleic acid donor sequence comprises a region having portions of nucleic acid homology to a target sequence.

In certain embodiments, the nucleic acid donor sequence is introduced into a target sequence by a homology-independent integration mechanism.

In certain embodiments, the homology-independent integration mechanism comprises engineering a cleavage site sequence into the nucleic acid donor, wherein the cleavage site sequence is also present in the target sequence.

In certain embodiments, a terminal block moiety is attached at the 5′ end of the nucleic acid donor sequence.

In certain embodiments, the terminal block moiety comprises one or more of ethylene glycol, polyethylene glycol (PEG), a polyamine having at least two amino groups, or an alkanediol.

In certain embodiments, the terminal block moiety comprises triethylene glycol or tetraethylene glycol.

In certain embodiments, a ssNA moiety is attached at the 5′ end of the nucleic acid donor sequence.

In certain embodiments, the ssNA moiety comprises a single stranded RNA (ssRNA).

In certain embodiments, the ssNA moiety or the ssRNA comprises a sequence selected from the group consisting of:
(mG)(mG)(mA)(mA)(mG)(mG)(mG)(mC)(mC)(mG)(mA)(mG)(mC)(mG)(mC);
(mG) #(mG) #(mA) #(mA)(mG)(mG)(mG)(mC)(mC)(mG)(mA)(mG)(mC)(mG)(mC);

(mG) #(mG) #(mA) #(mA) #(mG) #(mG) #(mG) #(mC) #(mC)(mG)(mA)(mG)(mC)(mG)(m C);
(mG) #(mG) #(mA) #(mA) #(mG) #(mG) #(mG) #(mC) #(mC) #(mG) #(mA) #(mG) #(mC) #(m G) #(mC) #;
(dG)(dG)(dA)(dA)(dG)(dG)(dG)(dC)(dC)(dG)(dA)(dG)(dC)(dG)(dC);
(dG) #(dG) #(dA) #(dA)(dG)(dG)(dG)(dC)(dC)(dG)(dA)(dG)(dC)(dG)(dC);
(dG) #(dG) #(dA) #(dA) #(dG) #(dG) #(dG) #(dC) #(dC)(dG)(dA)(dG)(dC)(dG)(dC);
(dG) #(dG) #(dA) #(dA) #(dG) #(dG) #(dG) #(dC) #(dC) #(dG) #(dA) #(dG) #(dC) #(dG) #(dC) #;
(mA)(mA)(mG)(mA)(mA)(mG)(mA)(mA)(mG)(mA)(mA)(mG)(mA)(mA)(mG);
(mU)(mU)(mC)(mU)(mU)(mC)(mU)(mU)(mC)(mU)(mU)(mC)(mU)(mU)(mC);
(dT) #(dT) #(dA) #(dG) #(dG) #(dG) #(dT) #(dT) #(dA) #(dG) #(dG) #(dG) #(dT) #(dT) #(dA) #(dG) #(dG) #(dG) #(dT) #(dT) #(dA) #(dG) #(dG) #(dG) #; or
(mU) #(mU) #(mA) #(mG) #(mG) #(mG) #(mU) #(mU) #(mA) #(mG) #(mG) #(mG) #(mU) #(mU) #(mA) #(mG) #(mG) #(mG) #(mU) #(mU) #(mA) #(mG) #(mG) #(mG) #,
wherein “m” corresponds to a nucleotide with a 2′-OMe modification; “d” corresponds to a nucleotide with a 2′H modification; and “#” corresponds to a phosphorothioate internucleotide linkage.

In certain embodiments, the ssNA moiety is attached to the 5′ end of the nucleic acid donor sequence with a linker.

In certain embodiments, the linker is selected from the group consisting of aminoethoxyethoxyacetate (AEEA), aminohexanoic acid, oligoglycine, ethylene glycol, polyethylene glycol (PEG), amino C6, and amino C12.

In certain embodiments, the linker comprises triethylene glycol or tetraethylene glycol.

In certain embodiments, the ssNA further comprises one or more of ethylene glycol, polyethylene glycol (PEG), a polyamine having at least two amino groups, and an alkanediol, attached to the 5′ end of the ssNA.

In certain embodiments, the ssNA further comprises one or more modified nucleotides.

In certain embodiments, the one or more modified nucleotides are selected from the group consisting of a 2′-O-alkyl modified nucleotide, a 2′-fluoro modified nucleotide, a nucleotide comprising a 5′-phosphorothioate group, a 2′-deoxy-modified nucleotide, a locked nucleic acid (LNA), a bridged nucleotide, a constrained nucleotide, a bicyclic nucleotide, an abasic nucleotide, a 2′-amino-modified nucleotide, a 2′-alkyl-modified nucleotide, a morpholino nucleotide, a phosphoramidate, a peptide nucleic acid, and a non-natural base comprising nucleotide.

In certain embodiments, the one or more modified nucleotides are 2′-O-methyl (2′-OMe) modified nucleotides.

In certain embodiments, the ssNA comprises one or more modified internucleotide linkages.

In certain embodiments, the modified internucleotide linkage comprises a phosphorothioate internucleotide linkage.

In certain embodiments, the modified internucleotide linkage comprises a modified internucleotide linkage of Formula I:

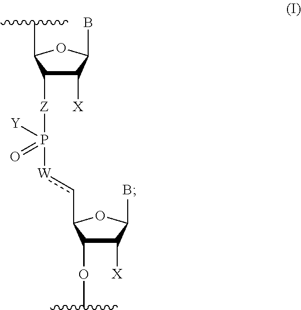

wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O, OH, OR, NH, NH₂, S—, and SH;
Z is selected from the group consisting of O and CH₂;
R is a protecting group; and
[symbol] is an optional double bond.

In certain embodiments, the ssNA is about 1 base in length to about 50 bases in length.

In certain embodiments, the ssNA is about 8 bases in length to about 30 bases in length.

In certain embodiments, the ssNA comprises 3, 4, 5, 6, 7, 8, 9, 10, 11, 12, 13, 14, 15, 16, 17, 18, 19, 20, 21, 22, 23, or 24 phosphorothioate internucleotide linkages.

In certain embodiments, every nucleotide in the ssNA comprises a phosphorothioate internucleotide linkages.

In certain embodiments, every nucleotide in the ssNA comprises a 2′-OMe modified nucleotide.

In certain embodiments, every nucleotide in the ssNA comprises a 2′-deoxy-modified nucleotide.

In certain embodiments, the ssNA comprises an immunosuppressive sequence.

In certain embodiments, the immunosuppressive sequence comprises (UUAGGG)ₙ or (TTAGGG)ₙ, wherein n is an integer from 1 to 5.

In certain embodiments, the ssNA binds to a terminal adaptor ligand.

In certain embodiments, the ssNA binds the terminal adaptor ligand through nucleic acid base-pairing interactions.

In certain embodiments, the terminal adaptor ligand comprises a peptide nucleic acid (PNA), a ssRNA, or a ssDNA.

In certain embodiments, the terminal adaptor ligand is attached to a terminal adaptor ligand moiety that confers one or more functionalities to the nucleic acid donor sequence.

In certain embodiments, the one or more functionalities is selected from the group consisting of tissue targeting, PK-modification, and nuclear localization.

In certain embodiments, the terminal adaptor ligand moiety is a peptide, a carbohydrate, a lipid, a steroid, or a small molecule.

In certain embodiments, the terminal adaptor ligand moiety is a nuclear localization signal (NLS).

In certain embodiments, the NLS comprises PKKKRK.

In certain embodiments, the terminal adaptor ligand is attached to the terminal adaptor ligand moiety through a linker.

In certain embodiments, the linker is selected from the group consisting of aminoethoxyethoxyacetate (AEEA), aminohexanoic acid, oligoglycine, ethylene glycol, PEG, amino C6, and amino C12.

In certain embodiments, the nucleic acid donor sequence is double-stranded.

In certain embodiments, the nucleic acid donor sequence is single-stranded.

In certain embodiments, the single stranded nucleic acid is a single stranded donor oligonucleotide (ssODN).

In certain embodiments, the nucleic acid donor sequence comprises portions of nucleic acid homology that are about 20 bases in length to about 1,000 bases in length.

In certain embodiments, the nucleic acid donor sequence comprises 1, 2, 3, 4, 5, 6, 7, 8, 9, 10, 11, 12, 13, 14, 15, 16, 17, 18, 19, or 20 phosphorothioate internucleotide linkages at the 5′ end.

In certain embodiments, the phosphorothioate internucleotide linkages are consecutive from the 5′ end.

In certain embodiments, the phosphorothioate internucleotide linkages are alternating from the 5′ end.

In certain embodiments, the nucleic acid donor sequence comprising the one or more phosphorothioate internucleotide linkages enhances donor genome integration relative to a nucleic acid donor sequence lacking the one or more phosphorothioate internucleotide linkages.

In certain embodiments, donor genome integration is enhanced about 2-fold or greater.

In certain embodiments, the nucleic acid donor sequence comprising the one or more phosphorothioate internucleotide linkages enhances donor genome integration in an immune cell or a hematopoietic stem or progenitor cell (HSPC) relative to a nucleic acid donor sequence lacking the one or more phosphorothioate internucleotide linkages.

In certain embodiments, donor genome integration is enhanced about 2-fold, about 3-fold, about 4-fold, about 5-fold, about 6-fold, about 7-fold, or greater.

In certain embodiments, the immune cell is a primary immune cell.

In certain embodiments, the primary immune cell is a T cell, a NK cell, or a B cell.

In one aspect, the disclosure provides a method of introducing a nucleic acid donor sequence into a target sequence of a genome in a cell, the method comprising: i) contacting the cell with the nucleic acid donor sequence recited above; and ii) contacting the cell with an agent that creates a double-stranded break at or near the target sequence.

In certain embodiments, the cell is contacted with the nucleic acid donor sequence prior to, simultaneously with, or after contacting the cell with the agent that creates a double-stranded break.

In certain embodiments, the contacting occurs in vitro, ex vivo, or in vivo.

In certain embodiments, the agent is a polypeptide, or a nucleic acid sequence encoding a polypeptide, selected from the group consisting of a zinc finger nuclease (ZFN), a transcription-activator like effector nuclease (TALEN), and an RNA-guided nuclease.

In one aspect, the disclosure provides a genome-editing system comprising: i) the nucleic acid donor sequence recited above; and ii) an agent that creates a double-stranded break at or near a target sequence.

In certain embodiments, the agent is a polypeptide, or a nucleic acid sequence encoding a polypeptide, selected from the group consisting of a zinc finger nuclease (ZFN), a transcription-activator like effector nuclease (TALEN), and an RNA-guided nuclease.

In one aspect, the disclosure provides a method of introducing a nucleic acid donor sequence into a target sequence of a genome in an immune cell or a hematopoietic stem or progenitor cell (HSPC), the method comprising: i) contacting the cell with a nucleic acid donor sequence comprising a ssNA moiety; and ii) contacting the cell with an agent that creates a double-stranded break at or near the target sequence.

In certain embodiments, the cell is contacted with the nucleic acid donor sequence prior to, simultaneously with, or after contacting the cell with the agent that creates a double-stranded break.

In certain embodiments, the contacting occurs in vitro, ex vivo, or in vivo.

In certain embodiments, the agent is a polypeptide, or a nucleic acid sequence encoding a polypeptide, selected from the group consisting of a zinc finger nuclease (ZFN), a transcription-activator like effector nuclease (TALEN), and an RNA-guided nuclease.

In certain embodiments, the ssNA comprises 1, 2, 3, 4, 5, 6, 7, 8, 9, 10, 11, 12, 13, 14, 15, 16, 17, 18, 19, 20, 21, 22, 23, or 24 phosphorothioate internucleotide linkages.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIG. 1 depicts homology-directed repair (HDR) fold change of several modified donor templates compared to an unmodified donor template in Jukat cells.

FIG. 2 depicts HDR fold change of several modified donor templates compared to an unmodified donor template in primary human T cells. For each of D1-D15, the left bar corresponds to a donor template dose of 0.1 pmol and the right bar corresponds to a donor template dose of 1 pmol.

### DETAILED DESCRIPTION

Gene editing technology holds much promise for personalized medicine, for example, to repair particular mutations or introduce exogenous genes that “rescue” the deleterious effects of a mutation. In essence, gene editing takes place in two steps: inducing a double-stranded break (DSBs) or other genetic lesion, e.g., using nucleases such as Cas9, and repairing the SSBs or DSBs by a repair mechanism. The current pre-clinical development of gene editing technology for therapeutic use can be generally categorized into two strategies: gene disruption that is protective (e.g., CCR4 disruption for HIV protection) and precise repairing of a known mutation. However, the efficacy of current methods for gene editing is hampered by the low frequency or efficiency of the repair mechanism.

“Homology-directed repair” or “HDR” is a mechanism to repair double stranded DNA breaks in cells. HDR generally relies on the process of homologous recombination, whereby stretches of nucleic acid sequence homology are used to repair the double stranded DNA break. During HDR, a strand of the homologous sequence of a nucleic acid donor invades, or hybridizes, with a resected portion of the cut DNA. A DNA polymerase, using the resected DNA as a primer, elongates the cut DNA, using the invaded donor sequence as a template. After elongation and break repair, the new sequence at the site of the cut possesses whatever sequence was present in the nucleic acid donor is used in the repair process. The process of HDR is further described in Jasin et al. (Cold Spring Harb. Perspect. Biol. 2013 November; 5(11): a012740), incorporated herein by reference.

“Homology-independent targeted integration” or “HITI” is a mechanism to integrate a nucleic acid donor sequence into the site of a double stranded break in cells. HITI utilizes the endogenous non-homologous end joining (NHEJ) mechanism to achieve the integration. A double stranded break is produced by a nuclease (e.g., CRISPR gene editing system comprising a CRISPR nuclease and guide RNA). The nucleic acid donor sequence is designed such that integration only occurs in the proper, forward direction. Integration events in the reverse direction will be cut again by the nuclease. Non-integration events will also be cut again by the nuclease. The process of HITI is further described in Suzuki et al. (Nature 2016 December; 540(7631): 144-149), incorporated herein by reference.

The methods and compositions of the disclosure address the limitations of current gene editing mechanisms (e.g., HDR or HITI) by providing novel modified nucleic acid donor templates which improve, for example, the efficiency, efficacy and/or precision of gene editing in vitro, ex vivo or in vivo. Without wishing to be bound by theory, the modified nucleic acid donor templates of the disclosure possess enhanced precision gene editing due to increased potency of the donor. Increased potency may be achieved through one or a combination of the following mechanisms: 1) resistance to engagement by enzymes that would metabolize nucleic acid donor ends, including, but not limited to, polymerases, nucleases, recombinases, and ligases; 2) increased nuclear localization and/or retention; 3) ligation resistance, which would leave more free ends of the nucleic acid donor available to serve as a template; 4) polymerase blocking, which may promote template switching; and/or 5) improved ability to engage repair machinery by making nucleic acid donor homology arms more accessible for strand invasion.

The compositions and methods may be used to repair mutations (e.g., compound heterozygous mutations) that are associated with certain diseases (e.g., monogenic recessive diseases). In some aspects, the modified nucleic acid donor templated may be employed with gene editing complexes (e.g., CRISPR/Cas system) to enable genome engineering at specific nucleotide positions in a homologous target nucleic acid of a host cell (e.g., homologous chromosomes that are compound heterozygous at a particular allele). In some aspects, the disclosure provides a method for targeted gene editing, the method comprising delivering to a cell (e.g., a cell of a disease subject) at least one component of a recombinant gene-editing complex together with the modified nucleic acid donor template, under conditions such that the recombinant gene editing complex induces a genetic lesion (e.g., nick or double stranded break) in a target site in the chromosome, and the donor template of the disclosure mediates a repair mechanism (e.g., HDR or HITI), thereby repairing the disease mutation.

Generally, nomenclature used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

As used herein, the terms “nucleic acid donor” or “nucleic acid donor template” or “donor template” or “donor sequence” or “donor” or “nucleic acid insert” or “insert” refer to any nucleic acid sequence, e.g., deoxyribonucleic acid, that may be used as a repair template in the repair mechanism (e.g., homology-directed repair (HDR) or homology independent targeted integration (HITI)). The nucleic acid donor may be double stranded or single stranded, e.g., double stranded DNA (dsDNA) or single stranded DNA (ssDNA). The nucleic acid donors of the disclosure may comprise varying polynucleotide lengths. In certain embodiments, the nucleic acid donor may be less than about 100 nucleotides in length, about 100 nucleotides in length, about 200 nucleotides in length, about 300 nucleotides in length, about 400 nucleotides in length, about 500 nucleotides in length, about 600 nucleotides in length, about 700 nucleotides in length, about 800 nucleotides in length, about 900 nucleotides in length, about 1000 nucleotides in length, or greater than about 1000 nucleotides in length. A nucleic acid donor of less than or equal to 200 nucleotides in length may also be referred to as a “short” nucleic acid donor. In certain embodiments, the nucleic acid donor is a single stranded donor oligonucleotide (ssODN). The nucleic acid donors to be inserted into the genome of a cell may be of any nucleotide length as needed by the skilled practitioner. For example, but in no way limiting, the nucleotide portion may be as short as a single nucleotide or greater than ten kilobases.

In one aspect, the disclosure provides an isolated nucleic acid donor sequence comprising a 5′ end and a 3′ end, wherein the nucleic acid donor sequence comprises one or more phosphorothioate internucleotide linkages at the 5′ end.

In certain embodiments, the nucleic acid donor sequence comprises 1, 2, 3, 4, 5, 6, 7, 8, 9, 10, 11, 12, 13, 14, 15, 16, 17, 18, 19, or 20 phosphorothioate internucleotide linkages at the 5′ end.

In certain embodiments, the phosphorothioate internucleotide linkages are consecutive from the 5′ end. In certain embodiments, the phosphorothioate internucleotide linkages are alternating from the 5′ end.

In an embodiment, the nucleic acid donors of the disclosure comprise a nucleotide sequence to be inserted into the genome of a cell, for example, an exogenous sequence to be inserted into the genome of a cell. The exogenous sequence may comprise a gene. The gene may encode for a protein, such as a therapeutic protein or a selectable marker protein. In certain embodiments, the selectable marker may encode for a selectable marker protein that confers resistance to an agent that reduces cell growth or causes cell death. Examples of such agents, included, but not limited to, ampicillin, blasticidin, bleomycin, chloramphenicol, gentamycin, hygromycin, kanamycin, lincomycin, methotrexate, neomycin, phosphinothricin, puromycin, tetracyclin, and zeocin. In other embodiments, the selectable marker may encode a fluorescent or luminescent protein (e.g., luciferase or GFP). The gene may be derived from the same species organism of the cell in which the gene is to be inserted. The gene may be derived from a different species organism of the cell in which the gene is to be inserted. The gene may be a chimeric sequence comprising sequences of multiple species. The nucleic acid donor may comprise a sequence that encodes for a non-coding RNA. Examples of non-coding RNAs include, but are not limited to, transfer RNAs (tRNAs), ribosomal RNAs (rRNAs), small RNAs such as siRNA, miRNA, piRNA, snoRNA, snRNA, exosomal RNA (exRNA). The nucleic acid donor may comprise a sequence that is not expressed. The nucleic acid donor may comprise a sequence that reduces or eliminates the expression of an endogenous gene in the cell.

In the case of HDR-mediated gene editing, the nucleic acid donors of the disclosure further comprise homology arms at the 5′ end and 3′ end, for example, a first and second homology arm. The homology arms are nucleic acid sequences that share sufficient homology with a target site in the genome of a cell to mediate HDR. Each homology arm may comprise varying polynucleotide lengths. It will be understood to those of skill in the art that homology arm nucleic acid sequences are an extension of the existing nucleic acid donor sequence as described above.

In certain embodiments the first homology arm may be about 20 nucleotides in length to about 1000 nucleotides in length. In certain embodiments the first homology arm may be less than about 20 nucleotides in length, about 20 nucleotides in length, about 30 nucleotides in length, about 40 nucleotides in length, about 50 nucleotides in length, about 60 nucleotides in length, about 70 nucleotides in length, about 80 nucleotides in length, about 90 nucleotides in length, about 100 nucleotides in length, about 200 nucleotides in length, about 300 nucleotides in length, about 400 nucleotides in length, about 500 nucleotides in length, about 600 nucleotides in length, about 700 nucleotides in length, about 800 nucleotides in length, about 900 nucleotides in length, about 1000 nucleotides in length, or greater than about 1000 nucleotides in length.

In certain embodiments the second homology arm may be about 20 nucleotides in length to about 1000 nucleotides in length. In certain embodiments the second homology arm may be less than about 20 nucleotides in length, about 20 nucleotides in length, about 30 nucleotides in length, about 40 nucleotides in length, about 50 nucleotides in length, about 60 nucleotides in length, about 70 nucleotides in length, about 80 nucleotides in length, about 90 nucleotides in length, about 100 nucleotides in length, about 200 nucleotides in length, about 300 nucleotides in length, about 400 nucleotides in length, about 500 nucleotides in length, about 600 nucleotides in length, about 700 nucleotides in length, about 800 nucleotides in length, about 900 nucleotides in length, about 1000 nucleotides in length, or greater than about 1000 nucleotides in length.

In certain embodiments, the first and second homology arm of the nucleic acid donor may comprise different nucleotide lengths. As an example for illustrative purposes, but in no way limiting, the homology arm at the 5′ end of the nucleic acid donor (the first homology arm) may be 100 nucleotides in length and the homology arm at the 3′ end of the nucleic acid donor (the second homology arm) may be 150 nucleotides in length.

### Single Strand Nucleic Acid Moiety

As used herein, the term “single strand nucleic acid moiety” or “ssNA moiety” refers to a heterologous 5′ end modification to the nucleic acid donor. In certain embodiments, the ssNA enhances HDR efficiency in an HDR assay as compared to a nucleic acid donor lacking the ssNA. In certain embodiments, the ssNA blocks or inhibits DNA polymerase extension, elongation and/or initiation (e.g., in a PCR assay). In certain embodiments, the ssNA is a ssRNA

In one aspect, the disclosure provides an isolated nucleic acid donor sequence comprising a 5′ end and a 3′ end, wherein a single-stranded RNA (ssNA) moiety is attached at the nucleic acid donor sequence 5′ end, wherein the ssNA comprises at least three phosphorothioate internucleotide linkages. It has been surprisingly discovered that the addition of phosphorothioate internucleotide linkages into the ssNA moiety and/or the nucleic acid donor sequence results in enhanced donor genome integration compared to nucleic acid donor sequences that do not have phosphorothioate internucleotide linkages.

In certain embodiments, the ssNA comprises 3, 4, 5, 6, 7, 8, 9, 10, 11, 12, 13, 14, 15, 16, 17, 18, 19, 20, 21, 22, 23, or 24 phosphorothioate internucleotide linkages.

In certain embodiments, every nucleotide in the ssNA comprises a phosphorothioate internucleotide linkage.

In another aspect, the disclosure provides an isolated nucleic acid donor sequence comprising a 5′ end and a 3′ end, wherein a single-stranded RNA (ssNA) moiety is attached at the nucleic acid donor sequence 5′ end, and wherein the ssNA comprises an immunosuppressive sequence.

As used herein, the term “immunosuppressive sequence” refers to a nucleic acid sequence that reduces an immune response. In certain embodiments, the immunosuppressive sequence reduces an immune response to the isolated nucleic acid donor sequence and/or an agent that creates a double-stranded break at or near a target sequence in a genome.

In certain embodiments, administration of the nucleic acid donor sequence comprising the immunosuppressive sequence to a subject elicits an inflammatory response in the subject that is at least 2-fold, at least 5-fold, at least 10-fold, or at least 50-fold lower than a nucleic acid donor sequence that does not comprise the immunosuppressive sequence.

In certain embodiments, the immunosuppressive sequence inhibits production of one or more proinflammatory cytokines.

In certain embodiments, the immunosuppressive sequence inhibits an inflammatory response induced by the toll-like receptor (TLR) pathway.

In certain embodiments, the immunosuppressive sequence comprises (UUAGGG)ₙ or (TTAGGG)ₙ, wherein n is an integer from 1 to 5.

In certain embodiments, the immunosuppressive sequence comprises UUAGGG. In certain embodiments, the immunosuppressive sequence comprises UUAGGGUUAGGG. In certain embodiments, the immunosuppressive sequence comprises UUAGGGUUAGGGUUAGGG. In certain embodiments, the immunosuppressive sequence comprises UUAGGGUUAGGGUUAGGGUUAGGG. In certain embodiments, the immunosuppressive sequence comprises UUAGGGUUAGGGUUAGGGUUAGGGUUAGGG.

In certain embodiments, the immunosuppressive sequence comprises TTAGGG. In certain embodiments, the immunosuppressive sequence comprises TTAGGGTTAGGG. In certain embodiments, the immunosuppressive sequence comprises TTAGGGTTAGGGTTAGGG. In certain embodiments, the immunosuppressive sequence comprises TTAGGGTTAGGGTTAGGGTTAGGG. In certain embodiments, the immunosuppressive sequence comprises TTAGGGTTAGGGTTAGGGTTAGGGTTAGGG.

In certain embodiments, the immunosuppressive sequence comprises (mU) #(mU) #(mA) #(mG) #(mG) #(mG) #(mU) #(mU) #(mA) #(mG) #(mG) #(mG) #(mU) #(mU) #(mA) #(mG) #(mG) #(mG) #(mU) #(mU) #(mA) #(mG) #(mG) #(mG) #, wherein “m” corresponds to a 2′-O-methyl (2′-OMe) modified nucleotide (i.e., a 2′-OMe modified U, A, G, or C nucleotide), and “#” corresponds to a phosphorothioate internucleotide linkage.

In certain embodiments, the immunosuppressive sequence comprises (dU) #(dU) #(dA) #(dG) #(dG) #(dG) #(dU) #(dU) #(dA) #(dG) #(dG) #(dG) #(dU) #(dU) #(dA) #(dG) #(dG) #(dG) #(dU) #(dU) #(dA) #(dG) #(dG) #(dG) #, wherein “d” corresponds to a 2′-deoxy-modified nucleotide (i.e., a 2′-deoxy modified U, A, G, or C nucleotide), and “#” corresponds to a phosphorothioate internucleotide linkage.

In certain embodiments, the immunosuppressive sequence is selected from the group consisting of:

GGGGGGGGGGGGGGGGGGGG,
TGCTCCTGGAGGGGTTGT,
TCCTGGAGGGGTTGT,
TGCTTGCAAGCTTGCAAGCA,
TTCCCATCCAGGCCTGGATGGGAA,
CCTGGATGGGAACTTACCGCTGCA,
CTTACCGCTGCACCTGGATGGGAA,
CCTGGATGGGAATTCCCATCCAGG,
TGACTGTGAAGGTTAGAGATGA,
CTCCTATTGGGGGTTTCCTAT,
TGGCGCGCACCCACGGCCTG,
CCTGGATGGGAA,
CCTGGCGGGG,
TCCTGGATGGGAAGT,
TCCTGGAGGGGAAGT,
and
TCCTGGCGGGGAAGT.

Additional immunosuppressive sequences are described further in Lenert (Mediators Inflamm., 2010, Article ID 986596), incorporated herein by reference.

In certain embodiments, the ssNA moiety is attached to the 5′ end of the nucleic acid donor sequence with a linker.

In certain embodiments, the linker is selected from the group consisting of aminoethoxyethoxyacetate (AEEA), aminohexanoic acid, oligoglycine, ethylene glycol, polyethylene glycol (PEG), amino C6, and amino C12. In certain embodiments, the linker comprises triethylene glycol or tetraethylene glycol.

In certain embodiments, the ssNA comprises one or more modified nucleotides.

In certain embodiments, the one or more modified nucleotides are selected from the group consisting of a 2′-O-alkyl modified nucleotide, a 2′-fluoro modified nucleotide, a nucleotide comprising a 5′-phosphorothioate group, a 2′-deoxy-modified nucleotide, a locked nucleic acid (LNA), a bridged nucleotide, a constrained nucleotide, a bicyclic nucleotide, an abasic nucleotide, a 2′-amino-modified nucleotide, a 2′-alkyl-modified nucleotide, a morpholino nucleotide, a phosphoramidate, a peptide nucleic acid, and a non-natural base comprising nucleotide.

In certain embodiments, the one or more modified nucleotides are 2′-O-methyl (2′-OMe) modified nucleotides.

In certain embodiments, every nucleotide in the ssNA comprises a 2′-OMe modified nucleotide.

In certain embodiments, every nucleotide in the ssNA comprises a 2′-deoxy-modified nucleotide.

In certain embodiments, the ssNA comprises one or more modified internucleotide linkages.

In certain embodiments, the modified internucleotide linkage comprises a phosphorothioate internucleotide linkage.

In certain embodiments, the modified internucleotide linkage comprises a modified internucleotide linkage of Formula I:

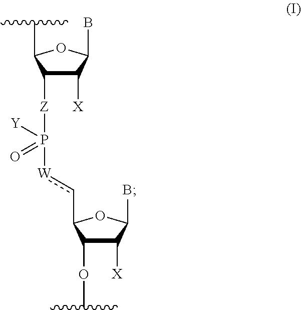

wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O, OH, OR, NH, NH₂, S—, and SH;
Z is selected from the group consisting of O and CH₂;
R is a protecting group; and
[symbol] is an optional double bond.

In certain embodiments, the ssNA comprises 3, 4, 5, 6, 7, 8, 9, 10, 11, 12, 13, 14, 15, 16, 17, 18, 19, 20, 21, 22, 23, or 24 phosphorothioate internucleotide linkages.

In certain embodiments, every nucleotide in the ssNA comprises a phosphorothioate internucleotide linkages.

In certain embodiments, every nucleotide in the ssNA comprises a 2′-OMe modified nucleotide.

In certain embodiments, every nucleotide in the ssNA comprises a 2′-deoxy-modified nucleotide.

The ssNA may comprise varying nucleotide lengths. Any length of ssNA necessary to enhance gene editing may be used. For example, a single RNA nucleotide may be sufficient as a terminal adaptor to enhance precision gene editing. Without being bound by theory, a single RNA nucleotide may act as a polymerase blocker, thus contributing to enhanced precision gene editing. In certain embodiments, the ssNA may be about 1-100 nucleotides in length. In certain embodiments, the ssNA may be about 1-50 nucleotides in length. In certain embodiments, the ssNA may be about 8-30 nucleotides in length. In certain embodiments, the ssNA may be about 10-20 nucleotides in length. In certain embodiments, the ssNA may be less than about 10 nucleotides in length, about 10 nucleotides in length, about 15 nucleotides in length, about 20 nucleotides in length, about 25 nucleotides in length, about 30 nucleotides in length, about 35 nucleotides in length, about 40 nucleotides in length, about 45 nucleotides in length, about 50 nucleotides in length, or greater than about 50 nucleotides in length.

### Terminal Block Moiety

As used herein, the term “terminal block moiety” refers to a heterologous non-single stranded nucleic acid 5′ end modification to the nucleic acid donor. In certain embodiments, the terminal block moiety enhances HDR efficiency in an HDR assay as compared to a nucleic acid donor lacking the terminal block moiety. In certain embodiments, the terminal block moiety blocks or inhibits DNA polymerase extension, elongation and/or initiation (e.g., in a PCR assay). In certain embodiments, the terminal block moiety may comprise a polyethylene glycol (PEG) chain containing multiple ethylene glycol units. In an embodiment, the PEG chain comprises 3 or 4 ethylene glycol units, also referred to as triethylene glycol or tetraethylene glycol, both of which may be abbreviated as TEG. In an embodiment, the PEG chain comprises 2-100 or more ethylene glycol units. In an embodiment, the PEG chain comprises 2-20, 20-40, 40-60, 60-80, 80-100, or 100 or more ethylene glycol units. In an embodiment, the PEG chain comprises 2, 3, 4, 5, 6, 7, 8, 9, 10, 11, 12, 13, 14, 15, 16, 17, 18, 19, or 20 ethylene glycol units. In an embodiment, the terminal block moiety may comprise a single ethylene glycol unit.

The terminal block moiety may comprise a polyamine having at least two amino groups, such as putrescine, spermidine, spermine, diamino ethylene, 1,4,8 triamino octane, and the like.

The terminal block moiety may comprise an alkanediol, where the diols may be germinal, vicinal, 1,3 diols, 1,4 diols, 2,4, diols and the like.

The terminal block moiety may be up to 18 atoms in length comprising moieties selected from alky, aryl, hydroxyl, alkoxy, ether, heteroaryl, phosphorous, alkylamino, guanidinyl, amidinyl, amide, ester, carbonyl, sulfide, disulfide, carbonyl, carbamate, phosphordiamidate, phosphorothioate, piperazine, phosphodiester, and heterocycly.

The terminal block moiety may comprise any one or more of the above recited terminal block moieties. The terminal block moiety may be “multivalent” or “multivalent terminal block moiety”. The multivalent terminal block moiety may comprise two or more terminal block moieties, linked together in tandem, and further linked to the homology arms of the nucleic acid donor. Moreover, the terminal block moiety may be combined with the ssNA moiety recited above.

In the case of a double stranded nucleic acid donor, e.g., a dsDNA donor template, terminal block moiety may be linked to the 5′ end of the top, or the sense strand, and the 5′ end of the bottom, or antisense strand. Alternatively, the terminal block moiety may be linked to only one of the two strands, for example the 5′ end of the top strand or the 5′ end of the bottom strand. In certain embodiments, only the 5′ end of the nucleic acid donor contains a terminal block moiety.

In the case of a single stranded nucleic acid donor, e.g., a ssDNA donor template, terminal block moieties may be linked to the 5′ end.

The terminal block moieties of the disclosure are operably linked, or attached, to the nucleic acid donor sequences, i.e., the first and/or second homology arm of the nucleic acid donor sequences. In certain embodiments, the terminal block moieties are covalently linked to the nucleic acid donor. In certain embodiments, the terminal block moieties are non-covalently linked to the nucleic acid donor. In certain embodiments, the terminal block moieties are linked to a modified 5′ terminal nucleotide of the nucleic acid donor. In certain embodiments, the terminal block moieties are linked to the 5′ phosphate of the terminal nucleotide of the nucleic acid donor. In certain embodiments, terminal block moieties are linked to the 2′ ribose of the terminal nucleotide of the nucleic acid donor. In certain embodiments, the terminal block moieties are linked to the nucleotide base of the terminal nucleotide of the nucleic acid donor.

### HDR Assays

The terminal adaptors of the disclosure serve to enhance HDR of the nucleic acid donors. The terminal adaptors may enhance HDR of the nucleic acid donors through one or a combination of the following ways: 1) resistance to engagement by enzymes that would metabolize nucleic acid donor ends, including, but not limited to, polymerases, nucleases, recombinases, and ligases; 2) increased nuclear localization and/or retention; 3) ligation resistance, which would leave more free ends of the nucleic acid donor available to serve as a template; 4) polymerase blocking, which may promote template switching; and/or 5) improved ability to engage repair machinery by making nucleic acid donor homology arms more accessible for strand invasion.

Terminal adaptors that enhance HDR of a nucleic acid donor may be screened by several assays known in the art. Panels of different terminal adaptors may be synthesized and attached to a nucleic acid donor sequence. The nucleic acid donor, encoding for a fluorescent protein, may be inserted into the genome of a cell through HDR. The fluorescence may then be monitored by microscopy to identify successful integration of the nucleic acid donor.

Terminal adaptors that enhance HDR of a nucleic acid donor may be screened with a modified version of the “traffic light” reporter (TLR) assay, as described in Certo et al. Nature Methods. 8: 671-676 (2011). In this assay, a double strand break is introduced into a non-functional GFP coding sequence followed by a frameshifted mCherry reporter. Imprecise repair via non-homologous end-joining (NHEJ) restores frame in a subset of indels, resulting in mCherry (red) fluorescence. Conversely, precisely templated repair via HDR of the same lesion results in GFP (green) fluorescence. Using flow cytometry, the percentage of cells expressing either GFP (HDR) or mCherry (NHEJ) among the total number of cells can be easily quantified. In this manner, panels of different terminal adaptors may be tested for their ability to enhance HDR by monitoring GFP positive cells in the above recited TLR assay. The HDR assays may be conducted in vivo, ex vivo, or in vitro.

In certain embodiments, the modified donor templates of the disclosure exhibit enhanced HDR efficiency in a HDR assay as compared to a suitable control (e.g., a donor template lacking the ssNA or phosphorothioate internucleotide linkages of the disclosure). In certain embodiments, the modified donor templates exhibit an improvement in HDR efficiency of at least 10% (e.g., 10%, 15%, 20%, 25%, 30%, 35%, 40%, 45%, 50%, 60%, 70%, 80%, 90%, 95% or more) as compared to the suitable control.

### Terminal Adaptor Ligands

As used herein, the term “terminal adaptor ligand” refers to an agent which binds to the terminal adaptor. In an embodiment, the terminal adaptor ligand binds to the terminal adaptor through nucleic acid base-pairing interactions between a single stranded oligonucleotide (e.g., ssRNA) terminal adaptor and the terminal adaptor ligand. The terminal adaptor ligand may comprise a polynucleotide sequence. The terminal adaptor ligand may comprise a polynucleotide sequence with one or more modified nucleotides. In an embodiment, the terminal adaptor ligand is a peptide nucleic acid (PNA) that is sufficiently complementary to the ssRNA terminal adaptor to bind. In an embodiment, the terminal adaptor ligand is also a ssRNA that is sufficiently complementary to the ssRNA terminal adaptor to bind. In an embodiment, the terminal adaptor ligand is a ssDNA that is sufficiently complementary to the ssRNA terminal adaptor to bind.

The terminal adaptor ligand may further comprise an additional moiety attached to the terminal adaptor ligand. The terminal adaptor ligand may be attached to the moiety via a linker. In certain embodiments, the linker is selected from the group consisting of aminoethoxyethoxyacetate (AEEA), aminohexanoic acid, oligo glycine, PEG, amino C6, and amino C12. In certain embodiments, the terminal adaptor ligand may be attached to the moiety without a linker.

### Terminal Adaptor Ligand Moieties

As used herein, the term “terminal adaptor ligand moiety” refers to a functional group either 1) attached to the terminal adaptor ligand or 2) directly attached to the nucleic acid donor sequence, that may be useful for conferring one or more additional functionalities to the nucleic acid donor template. Functionalities include, but are not limited to, tissue targeting, PK-modification, and/or nuclear localization. The moiety may be a peptide, a carbohydrate, a lipid, a steroid, or a small molecule. In addition to one or more of the functionalities listed above, when the terminal adaptor ligand moiety is attached directly to the nucleic acid donor without the terminal adaptor ligand, the terminal adaptor ligand moiety may also enhance HDR of the nucleic acid donor.

In one embodiment, the moiety has an affinity for low density lipoprotein and/or intermediate density lipoprotein. In a related embodiment, the moiety is a saturated or unsaturated moiety having fewer than three double bonds.

In another embodiment, the moiety has an affinity for high density lipoprotein. In a related embodiment, the moiety is a polyunsaturated moiety having at three or more double bonds (e.g., having three, four, five, six, seven, eight, nine or ten double bonds). In a particular embodiment, the moiety is a polyunsaturated moiety having three double bonds. In a particular embodiment, the moiety is a polyunsaturated moiety having four double bonds. In a particular embodiment, the moiety is a polyunsaturated moiety having five double bonds. In a particular embodiment, the moiety is a polyunsaturated moiety having six double bonds.

In another embodiment, the moiety is selected from the group consisting of fatty acids, steroids, secosteroids, lipids, gangliosides and nucleoside analogs, and endocannabinoids.

In another embodiment, the moiety is a neuromodulatory lipid, e.g., an endocannabinoid. Non-limiting examples of endocannabinoids include: anandamide, arachidonoylethanolamine, 2-Arachidonyl glyceryl ether (noladin ether), 2-Arachidonyl glyceryl ether (noladin ether), 2-Arachidonoylglycerol, and N-Arachidonoyl dopamine.

In another embodiment, the moiety is an omega-3 fatty acid. Non-limiting examples of omega-3 fatty acids include: hexadecatrienoic acid (HTA), alpha-linolenic acid (ALA), stearidonic acid (SDA), cicosatrienoic acid (ETE), cicosatetraenoic acid (ETA), cicosapentaenoic acid (EPA, timnodonic acid), hencicosapentaenoic acid (HPA), docosapentaenoic acid (DPA, clupanodonic acid), docosahexaenoic acid (DHA, cervonic acid), tetracosapentaenoic acid, and tetracosahexaenoic acid (nisinic acid).

In another embodiment, the moiety is an omega-6 fatty acid. Non-limiting examples of omega-6 fatty acids include: linoleic acid, gamma-linolenic acid (GLA), eicosadienoic acid, dihomo-gamma-linolenic acid (DGLA), arachidonic acid (AA), docosadienoic acid, adrenic acid, docosapentaenoic acid (osbond acid), tetracosatetraenoic acid, and tetracosapentaenoic acid.

In another embodiment, the moiety is an omega-9 fatty acid. Non-limiting examples of omega-9 fatty acids include: oleic acid, eicosenoic acid, mead acid, erucic acid, and nervonic acid.

In another embodiment, the moiety is a conjugated linolenic acid. Non-limiting examples of conjugated linolenic acids include: α-calendic acid, β-calendic acid, jacaric acid, α-eleostearic acid, β-eleostearic acid, catalpic acid, and punicic acid.

In another embodiment, the moiety is a saturated fatty acid. Non-limiting examples of saturated fatty acids include: caprylic acid, capric acid, docosanoic acid, lauric acid, myristic acid, palmitic acid, stearic acid, arachidic acid, behenic acid, lignoceric acid, and cerotic acid.

In another embodiment, the moiety is an acid selected from the group consisting of: rumelenic acid, α-parinaric acid, β-parinaric acid, bosseopentaenoic acid, pinolenic acid, and podocarpic acid.

In another embodiment, the moiety is selected from the group consisting of: docosanoic acid (DCA), docosahexaenoic acid (DHA), and eicosapentaenoic acid (EPA).

In another embodiment, the moiety is a secosteroid. In a particular embodiment, the moiety is calciferol.

In another embodiment, the moiety is an alkyl chain, a vitamin, a peptide, or a bioactive conjugate (including but not limited to: glycosphingolipids, polyunsaturated fatty acids, secosteroids, steroid hormones, sterol lipids and the like).

In another embodiment, the moiety is a lipophilic moiety selected from the group consisting of cholesterol, vitamin E, vitamin K, vitamin A, folic acid, cholic acid, adamantane acetic acid, 1-pyrene butyric acid, dihydrotestosterone, 1,3-Bis-O(hexadecyl)glycerol, geranyloxyhexyl group, hexadecylglycerol, borncol, menthol, 1,3-propanediol, heptadecyl group, palmitic acid, myristic acid, 03-(oleoyl)lithocholic acid, O3-(oleoyl)cholenic acid, dimethoxytrityl, or phenoxazine.

The moiety attached to the terminal adaptor ligand may be useful for cell or tissue targeting. Moieties useful for targeting include, but are not limited to, a thyrotropin, melanotropin, lectin, glycoprotein, surfactant protein A, mucin carbohydrate, multivalent lactose, multivalent galactose, N-acetyl-galactosamine (GalNAc), N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g., cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borncol, menthol, 1,3-propanediol, heptadecyl group, palmitic acid, stearic acid (e.g., glyceryl distearate), oleic acid, myristic acid, 03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG, MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu3+complexes of tetraazamacrocycles), dinitrophenyl, HRP or AP.

In certain embodiments, the moiety is a nuclear localization signal (NLS). Any NLS known in the art may be used as a moiety linked to the terminal adaptor ligand. In certain embodiments, the NLS comprises the peptide sequence of PKKKRK. Additional examples of NLSs may be found in Kosugi et al. J. Biol. Chem. 284: 478-485 (2009) and Bernhofer et al. Nucleic Acids Research 46: D503-D508 (2017).

### Modified Nucleotides

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

In certain embodiments, the above recited modified nucleotides may be incorporated into the nucleic acid donor sequences of the disclosure. In certain embodiments, the above recited modified nucleotides may be incorporated into the terminal adaptors and/or terminal adaptor ligands of the disclosure.

### Gene Editing Complexes

As used herein, “gene editing complex” refers to a biologically active molecule (e.g., a protein, one or more proteins, a nucleic acid, one or more nucleic acids, or any combination of the foregoing) configured for adding, disrupting, or changing genomic sequences (e.g., a gene sequence) by causing a genetic lesion (e.g., double stranded break (DSB)) in a target DNA or other target nucleic acid. In certain embodiments, the gene editing complex may further comprise the modified DNA donor templates of the disclosure, e.g., to enhance the efficacy of gene editing at the site of the genetic lesion in the genome of a cell. The genetic lesion (e.g., DSB) may be introduced in a number of ways known in the art. Examples of gene editing complexes include but are not limited nucleases such as transcription activator-like effector nucleases (TALENs), zinc finger nucleases (ZFNs), engineered meganuclease re-engineered homing endonucleases, the CRISPR/Cas system, and meganucleases (e.g., Meganuclease I-Scel). In some embodiments, a gene editing complex comprises proteins or molecules (e.g., components) related to the CRISPR/Cas system, including but not limited to Cas9, Cas6, dCas9, CRISPR RNA (crRNA), trans-activating crRNA (tracrRNA), and variants thereof. In some embodiments, the Cas protein is a Cpf1 protein, or a variant thereof.

In certain embodiments, the gene editing complex comprises a nuclease that introduces a double-stranded break (DSB) to facilitate gene editing. However, it will be appreciated that the gene editing complex may be configured to introduce single stranded nicks or single stranded breaks (SSBs) at the target site in the genome of a cell. For example, two nucleases may be used to introduce two SSBs at two adjacent target sites in the genome of a cell. By introducing two adjacent SSBs, a double stranded break is created.

As used herein, the terms “endonuclease” and “nuclease” refer to an enzyme that cleaves a phosphodiester bond or bonds within a polynucleotide chain. Nucleases may be naturally occurring or genetically engineered. Genetically engineered nucleases are particularly useful for genome editing and are generally classified into four families: zinc finger nucleases (ZFNs), transcription activator-like effector nucleases (TALENs), meganucleases (e.g., engineered meganucleases) and RNA guides nucleases such as the CRISPR-associated proteins (Cas nucleases).

### Meganucleases

A meganuclease, such as a homing endonuclease, refers to a double-stranded endonuclease having a polynucleotide recognition site of 14-40 base pairs, which can be either monomeric or dimeric. Meganucleases can be designed and predicted according to the procedures in US 2014/0121115 can be used in the present methods. A “custom-made meganuclease” refers to a meganuclease derived from a parental meganuclease that possesses recognition and/or cleavage that is altered from the parental meganuclease. Exemplary meganucleases include, but are not limited to, I-Sce I, I-Chu I, I-Dmo I, I-Cre I, I-Csm I, PI-Sce I, PI-Tli I, PI-Mtu I, I-Ceu I, I-Sce II, I-Sce III, HO, PI-Civ I, PI-Ctr I, PI-Aac I, PI-Bsu I, PI-Dha I, PI-Dra I, PI-Mav I, PI-Mch I, PI-Mfu I, PI-Mfl I, PI-Mga I, PI-Mgo I, PI Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, and PI-Tsp I; I-Sce I, I-Chu I, I-Dmo I, I-Cre I, I-Csm I, PI-Sce I, PI-Pfu I, PI-Tli I, PI-Mtu I, and I-Ceu I; I-Dmo I, I-Cre I, PI-Sce I, and PI-Pfu I Homing endonucleases generally cleave their DNA substrates as dimers, and do not have distinct binding and cleavage domains.

### Zinc Finger Nucleases

Zinc finger nucleases (ZFNs) are enzymes having a DNA cleavage domain and a DNA binding zinc finger domain. ZFNs may be made by fusing the nonspecific DNA cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR).

Zinc finger proteins can be designed and predicted according to the procedures in WO 98/54311, U.S. Pat. Nos. 9,187,758, 9,206,404 and 8,771,985 can be used in the present methods. WO 98/54311 discloses technology which allows the design of zinc finger protein domains that bind specific nucleotide sequences that are unique to a target gene. It has been calculated that a sequence comprising 18 nucleotides is sufficient to specify a unique location in the genome of higher organisms. Typically, therefore, the zinc finger protein domains are hexadactyl, i.e., contain 6 zinc fingers, each with its specifically designed alpha helix for interaction with a particular triplet. However, in some instances, a shorter or longer nucleotide target sequence may be desirable. Thus, the zinc finger domains in the proteins may contain at least 3 fingers, or from 2-12 fingers, or 3-8 fingers, or 3-4 fingers, or 5-7 fingers, or even 6 fingers. In one aspect, the ZFP contains 3 zinc fingers; in another aspect, the ZFP contains 4 zinc fingers. Additional description on ZFNs and their design for genome editing may be found in U.S. Pat. No. 20,120,329067A1, incorporated herein by reference.

### Transcription Activator Like Effector Nucleases (TALENs)

Transcription Activator-Like Effector Nucleases (TALENs) are artificial restriction enzymes generated by fusing the TAL effector DNA binding domain to a DNA cleavage domain. These reagents enable efficient, programmable, and specific DNA cleavage and represent powerful tools for genome editing in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, and U.S. Pat. No. 9,393,257, all of which are incorporated by reference herein in their entireties.

TAL effectors are proteins secreted by Xanthomonas bacteria. The DNA binding domain contains a highly conserved 33-34 amino acid sequence with hypervariable 12th and 13th amino acids. These two locations are highly variable (repeat variable di-residue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of the Fok1 endonuclease can be used to construct hybrid nucleases that are active in a yeast assay. These reagents are also active in plant cells and in animal cells. Initial TALEN studies used the wild-type Fok1 cleavage domain, but some subsequent TALEN studies also used Fok1 cleavage domain variants with mutations designed to improve cleavage specificity and cleavage activity. The Fok1 domain functions as a dimer, requiring two constructs with unique DNA binding domains for sites in the target genome with proper orientation and spacing. Both the number of amino acid residues between the TALEN DNA binding domain and the Fok 1 cleavage domain and the number of bases between the two individual TALEN binding sites are parameters for achieving high levels of activity. The number of amino acid residues between the TALEN DNA binding domain and the Fok1 cleavage domain may be modified by introduction of a spacer (distinct from the spacer sequence) between the plurality of TAL effector repeat sequences and the Fok1 endonuclease domain. The spacer sequence may be 12 to 30 nucleotides.

The relationship between amino acid sequence and DNA recognition of the TALEN binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two-step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms. In this manner, they can be used to correct mutations in the genome which, for example, cause disease.

In certain embodiments, the TALEN is a MegTALEN or MegaTAL. MegaTALs are fusion proteins that combine homing endonucleases with modular DNA binding domains of TALENs, resulting in improved DNA sequence targeting and increased gene editing efficiencies. N-terminal fusions of TAL anchors can be employed to increase the specificity and activity of a gene-targeted endonuclease, including one or more homing endonucleases such as one or more of the I-HjeMI, I-CpaMI, and I-Onul homing endonucleases. MegaTALs can be constructed using the Golden Gate assembly strategy described by Cermak et al, Nucl. Acids Res. 39:082-c82 (2011), using, e.g., an RVD plasmid library and destination vector. MegaTALs can be designed and predicted according to the procedures in WO 2013/126794 and WO 2014/191525 can be used in the present methods.

### RNA-Guide Nucleases

RNA-guided nucleases according to the present disclosure include, without limitation, naturally-occurring Class II CRISPR nucleases such as Cas9 (Type II) or Cas12a/Cpf1 (Type V), as well as other nucleases derived or obtained therefrom. Exemplary Cas9 nucleases that may be used in the present disclosure include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9). In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity).

Various RNA-guided nucleases may require different sequential relationships between PAMs and protospacers. In general, Cas9s recognize PAM sequences that are 5′ of the protospacer as visualized relative to the top or complementary strand. In addition to recognizing specific sequential orientations of PAMs and protospacers, RNA-guided nucleases generally recognize specific PAM sequences. S. aureus Cas9, for example, recognizes a PAM sequence of NNGRRT, wherein the N sequences are immediately 3′ of the region recognized by the gRNA targeting domain. S. pyogenes Cas9 recognizes NGG PAM sequences. It should also be noted that engineered RNA-guided nucleases can have PAM specificities that differ from the PAM specificities of similar nucleases (such as the naturally occurring variant from which an RNA-guided nuclease is derived, or the naturally occurring variant having the greatest amino acid sequence homology to an engineered RNA-guided nuclease). Modified Cas9s that recognize alternate PAM sequences are described below.

RNA-guided nucleases are also characterized by their DNA cleavage activity: naturally-occurring RNA-guided nucleases typically form DSBs in target nucleic acids, but engineered variants have been produced that generate only SSBs (discussed above; see also Ran et al. Nature Protocols, 8(11): 2281-2308 (2013), incorporated by reference herein), or that do not cut at all.

RNA-guided nucleases include nickase variants, such as a Cas9 nickase. Various RNA-guided nickases or CRISPR nickases are known in the art, such as an S. pyogenes Cas9 with a D10A mutation. A dual-nickase approach may be employed, wherein two nicks on opposite strands of a sequence of interest that are offset by one or more nucleotides are introduced. When the two nicks are introduced, a double stranded break is created.

Accordingly, one of skill in the art would be able to select the appropriate nuclease for the present disclosure.

### Guide RNA

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, e.g., 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or target polynucleotide sequence” is the DNA sequence in a genome of a cell that is complementary to the guide sequence of the gRNA.

In addition to the targeting domains, gRNAs typically include a plurality of domains that influence the formation or activity of gRNA/Cas9 complexes. For example, as mentioned above, the duplexed structure formed by first and secondary complementarity domains of a gRNA (also referred to as a repeat: anti-repeat duplex) interacts with the recognition (REC) lobe of Cas9 and may mediate the formation of Cas9/gRNA complexes (Nishimasu et al. Cell 156: 935-949 (2014); Nishimasu et al. Cell 162(2), 1113-1126 (2015), both incorporated by reference herein). It should be noted that the first and/or second complementarity domains can contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for example through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are necessary for nuclease activity in vivo but not necessarily in vitro (Nishimasu 2015, supra). A first stem-loop near the 3′ portion of the second complementarity domain is referred to variously as the “proximal domain,” “stem loop 1” (Nishimasu 2014, supra; Nishimasu 2015, supra) and the “nexus” (Briner 2014, supra). One or more additional stem loop structures are generally present near the 3′ end of the gRNA, with the number varying by species: S. pyogenes gRNAs typically include two 3′ stem loops (for a total of four stem loop structures including the repeat: anti-repeat duplex), while S. aureus and other species have only one (for a total of three). A description of conserved stem loop structures (and gRNA structures more generally) organized by species is provided in Briner 2014, which is incorporated herein by reference. Additional details regarding guide RNAs generally may be found in WO2018026976A1, which is incorporated herein by reference.

The RNA-guided nucleases may be combined with guide RNAs to form a genome-editing system. The RNA-guided nucleases may be combined with the guide RNAs to form a ribonucleoprotein (RNP) complex that may be delivered to a cell where genome-editing is desired. The RNA-guided nucleases and guide RNAs may be expressed in a cell where genome-editing is desired. For example, the RNA-guided nucleases and guide RNAs may be expressed from one or more polynucleotides such as a vector. The vector may be a viral vector, including, be not limited to, an adeno-associated virus (AAV) vector or a lentivirus (LV) vector. The RNA-guided nuclease may alternatively be expressed from a synthetic mRNA.

### Methods of Editing

The modified nucleic acid donor sequences disclosed herein display enhanced gene editing (i.e., enhanced donor genome integration) compared to an unmodified nucleic acid donor sequence. Surprisingly, modified nucleic acid donor sequences with phosphorothioate internucleotide linkages had enhanced gene editing compared to a nucleic acid donor sequence without a phosphorothioate internucleotide linkage. The phosphorothioate internucleotide linkages can be present in the ssNA moiety described above (i.e., 1 or more phosphorothioate internucleotide linkages). Separately or in addition, the phosphorothioate internucleotide linkages can be present in the 5′ end of the nucleic acid donor sequence (i.e., 1 or more phosphorothioate internucleotide linkages).

In one aspect, the disclosure provides a method of introducing a nucleic acid donor sequence into a target sequence of a genome in a cell, the method comprising: i) contacting the cell with the nucleic acid donor sequence recited above; and ii) contacting the cell with an agent that creates a double-stranded break at or near the target sequence.

In certain embodiments, the cell is contacted with the nucleic acid donor sequence prior to, simultaneously with, or after contacting the cell with the agent that creates a double-stranded break.

In certain embodiments, the contacting occurs in vitro, ex vivo, or in vivo.

In certain embodiments, the agent is a polypeptide, or a nucleic acid sequence encoding a polypeptide, selected from the group consisting of a zinc finger nuclease (ZFN), a transcription-activator like effector nuclease (TALEN), and an RNA-guided nuclease.

In certain embodiments, the nucleic acid donor sequence enhances donor genome integration in the cell relative to a nucleic acid donor sequence lacking the ssNA and/or phosphorothioate internucleotide linkages.

In certain embodiments, donor genome integration is enhanced about 2-fold, about 3-fold, about 4-fold, about 5-fold, about 6-fold, about 7-fold, or greater.

In certain embodiments, the cell is an immune cell or a hematopoietic stem or progenitor cell (HSPC). In certain embodiments, the immune cell is a primary immune cell (e.g., a human primary immune cell). In certain embodiments, the primary immune cell is a T cell, a NK cell, or a B cell.

In certain embodiments, the HSPC is isolated from bone marrow of a subject.

In another aspect, the disclosure provides a method of introducing a nucleic acid donor sequence into a target sequence of a genome in an immune cell or a hematopoietic stem or progenitor cell (HSPC), the method comprising: i) contacting the cell with a nucleic acid donor sequence comprising a ssNA moiety; and ii) contacting the cell with an agent that creates a double-stranded break at or near the target sequence.

In certain embodiments, the cell is contacted with the nucleic acid donor sequence prior to, simultaneously with, or after contacting the cell with the agent that creates a double-stranded break.

In certain embodiments, the contacting occurs in vitro, ex vivo, or in vivo.

In certain embodiments, the agent is a polypeptide, or a nucleic acid sequence encoding a polypeptide, selected from the group consisting of a zinc finger nuclease (ZFN), a transcription-activator like effector nuclease (TALEN), and an RNA-guided nuclease.

In certain embodiments, nucleic acid donor sequence enhances donor genome integration in an immune cell or a hematopoietic stem or progenitor cell (HSPC) relative to a nucleic acid donor sequence lacking the ssNA.

In certain embodiments, donor genome integration is enhanced about 2-fold, about 3-fold, about 4-fold, about 5-fold, about 6-fold, about 7-fold, or greater.

In certain embodiments, the immune cell is a primary immune cell.

In certain embodiments, the primary immune cell is a T cell, a NK cell, or a B cell.

In certain embodiments, the HSPC is isolated from bone marrow of a subject.

### Viral Delivery

The nucleic acid donors of the disclosure may be packaged in viral vector for delivery to a cell. Packaging of the nucleic acid donors may be achieved by annealing a ssRNA terminal adaptor to the viral genome for internal packaging inside the viral capsid. In a certain additional or alternative embodiments, the viral genome may encode for the components of the gene editing complex (e.g., RNA-guided nuclease and/or a guide RNA).

In some embodiments, the viral vector is an isolated recombinant adeno-associated virus (rAAV). As used herein with respect to AAVs, the term “isolated” refers to an AAV that has been artificially produced or obtained. Isolated AAVs may be produced using recombinant methods. Such AAVs are referred to herein as “recombinant AAVs.” In exemplary embodiments, recombinant AAVs (rAAVs) have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected. Methods for obtaining recombinant AAVs having a desired capsid protein are well known in the art. (See, for example, US 2003/0138772, the contents of which are incorporated herein by reference in their entirety). Typically, the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner. In some embodiments, a terminally grafted nuclease (e.g., at least one component of a gene editing complex) is present on all three capsid proteins (e.g., VP1, VP2, VP3) of a rAAV. In some embodiments, the terminally grafted nuclease is present on two of the capsid proteins (e.g., VP2 and VP3) of a rAAV. In some embodiments, the terminally grafted nuclease is present on a single capsid protein of a rAAV. In some embodiments, the terminally grafted nuclease is present on the VP2 capsid protein of the rAAV.

In some aspects, the instant disclosure relates to the location within an AAV capsid protein where a component of the disclosure (e.g., the nucleic acid donor template and/or at least one component of a gene editing complex) is grafted. In some embodiments, the component is N-terminally grafted to the capsid protein. In some embodiments, the component is C-terminally grafted to a capsid protein. In some embodiments, the component resides within the viral particle, and the viral particle does not contain a genome, e.g., a nucleic acid harboring a transgene.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art.

Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art. The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. In exemplary embodiments, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both of which are incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

The foregoing methods for packaging recombinant vectors in desired AAV capsids to produce the rAAVs of the disclosure are not meant to be limiting and other suitable methods will be apparent to the skilled artisan.

### Synthesis of Modified Nucleic Acid Donor Templates

The nucleic acid donors of the disclosure may be synthesized using standard molecular biology techniques known in the art. Double-stranded DNA donors may be synthesized by PCR. The donor sequence with homolog arms may be present in a vector. Oligonucleotide primers, synthesized to contain the ssNA moiety or terminal block moiety, are then used in a PCR reaction to generate the modified dsDNA donors. As an example, but in no way limiting, the oligonucleotide primers may be PEGylated, have a ssRNA at the 5′ end, or both.

Single strand DNA donors may be synthesized through reverse transcription. An RNA template may be used in combination with a reverse transcription oligonucleotide primer, synthesized to contain the terminal adaptors.

Single stranded donor oligonucleotides (ssODN), which may be shorter in length than a long ssDNA donor, may be synthesized directly to contain the terminal adaptors.

As an alternative mechanism to PCR or reverse transcription, modified nucleic acid donors of the disclosure may be synthesized by ligating the terminal adaptors to the unmodified nucleic acid donor. As an example, but in no way limiting, a vector containing the nucleic acid donor and homology arms may be cut by one or more restriction enzymes to linearize the vector. The terminal adaptors may then be ligated to the ends of the linearized vector.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1—Generation of Donor Nucleic Acid Templates

The methods recited herein were employed to generate the donor DNA templates used in the below recited Examples and Figures.

### Synthesis of PEGylated Oligonucleotides

PEG-modified oligonucleotides were synthesized using standard phosphoramidite methods on an ABI 394 synthesizer. Phosphoramidites were purchased from ChemGenes. Coupling times for 2′OMe-RNA and spacer phosphoramidites were extended to 5 minutes. Oligonucleotides were deprotected in concentrated aqueous ammonia at 55° C. for 16 hours. Oligonucleotides were desalted using either Nap-10 (Sephadex) columns or Amicon ultrafiltration. All the PEG-modified oligonucleotides were characterized on an Agilent 6530 Q-TOF LC/MS system with electrospray ionization.

PCR dsDNA Donor Generation

Donor template sequences with the homology arms and the desired insert for knock-in were generated by PCR. PCR products were cloned into ZeroBlunt TOPO vector (Invitrogen, #450245) and plasmids were purified using Macherey-Nagel midi-prep kits (cat #740412.50). Using the purified plasmids as templates and modified oligonucleotides as primers, donor sequences were PCR amplified with Phusion polymerase (NEB, #M0530S) or Q5 polymerase. The resulting modified PCR products were excised from 0.8-1% TAE agarose gel and purified using spin-columns. PCR conditions were optimized for each primer set with a gradient for the annealing temperature [1) 98° C. for 1:00 minute, 2) 98° C. for 15 seconds, 3) 50° C. to 64° C. for 30 seconds (choose optimal), 4) 72° C. for 1:00 minute (34 cycles), 5) 72° C. for 5:00 minutes, 6) 4° C. forever]. The modified oligonucleotides were designed to introduce the desired chemical modification into the donor templates (e.g., 2′-OMe modifications, 2′-H (DNA) modifications and phosphorothioate modifications). For example, but in no way limiting, a PEGylated oligonucleotide was used to introduce a PEG moiety into the donor template. Likewise, phosphorothioate internucleotide linkages were introduced at specific positions in the donor template using phosphorothioate-modified oligonucleotides. This method of introducing phosphorothioate internucleotide linkages avoids the stochastic placement caused by rolling circle amplification (RCA).

### Example 2—HDR Efficiency in Immune Cells

Several modified donor templates were tested in a screen to determine the impact of various modifications on HDR efficacy in immune cells. Single stranded RNA (ssRNA) sequences were attached to the 5′ ends of a double stranded DNA donor template with a PEG linker (either tricthylene glycol (TriEG) or tetraethylene glycol (tetraEG)). The ssRNA were modified with 2′ OMe, 2′ H, and/or phosphorothioate (PS) internucleotide linkages. For one modified donor template, the double stranded DNA donor was modified with 3 PS internucleotide linkages at the 5′ ends of each strand. In addition to the modified ssRNA, different ssRNA base sequences were used. Two different immunosuppressive sequences, a purine-rich sequence, and a pyrimidine-rich sequence were used. The ssRNA sequences are recited below in Table 1.

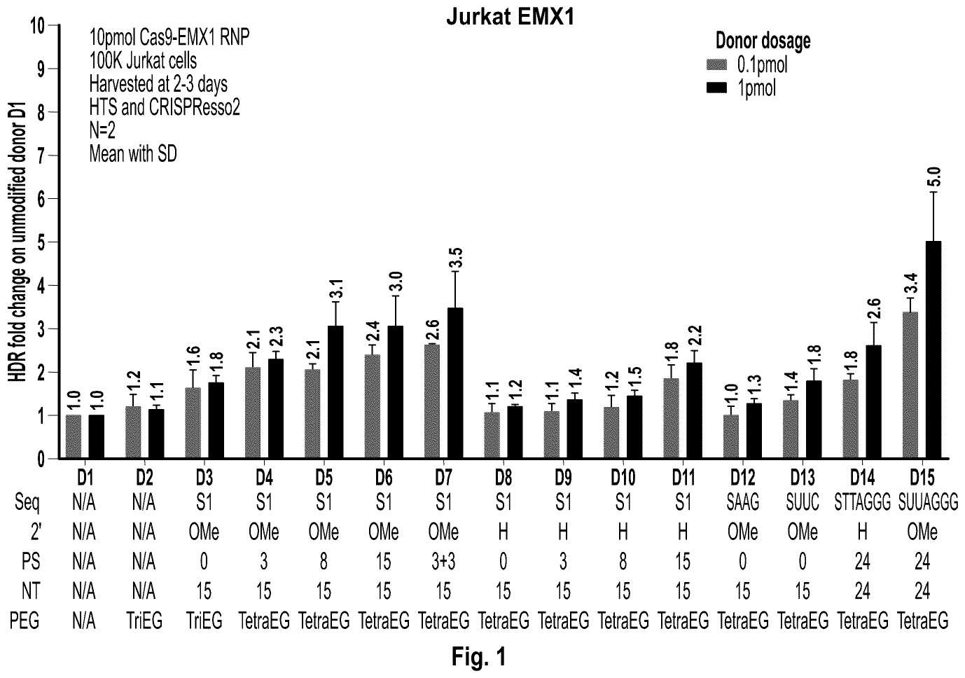

The donor templates were designed, from 5′ to 3′, as 85 bp homology arm (HA): 8 bp insertion: 91 bp HA. The 8 bp insertion was to replace an 11 bp region on the EMX1 locus by HDR. Ribonucleoprotein (RNP) complexes containing a Cas9 protein and a guide RNA targeting the EMX1 locus were prepared. The donors and RNPs were electroporated into Jurkat cells and genomic DNA was harvested 2-3 days post electroporation. 10 pmol of RNP was used, the donor template was used at either 0.1 pmol or 1 pmol, and 100,000 Jurkat cells were electroporated. A high throughput sequencing (HTS) library was prepared with the genomic DNA and the library was subjected to Illumina Miniseq platform for HTS, and the HTS data was analyzed by CRISPResso2 program. As shown in FIG. 1, numerous modified donors with ssRNA had higher HDR rates than the unmodified control template (D1). Increasing amounts of PS internucleotide linkages lead to increased HDR efficiency. A donor comprising a ssRNA with 3 PS internucleotide linkages linked to a double stranded DNA donor template also comprising 3 PS internucleotide linkages at the 5′ end demonstrated high level of HDR efficiency as well. In FIG. 1, the term “S1” refers to the ssNA attached to the 5′ end of the donor template with the sequence (mG)(mG)(mA)(mA)(mG)(mG)(mG)(mC)(mC)(mG)(mA)(mG)(mC)(mG)(mC) (D3 in FIG. 1), where “m” corresponds to a nucleotide with a 2′-OMe modification. This sequence is further modified as described in Table 1. In FIG. 1, “PEG” represents that type of PEG moiety between the ssNA and the donor template. “TriEG” corresponds to a triethylene glycol at the 3′ end of the ssNA and the 5′ end of the donor template. “TetraEG” corresponds to a tetraethylene glycol at the 3′ end of the ssNA and the 5′ end of the donor template.

The effect of ssNA sequence identity and length on HDR efficiency was also tested in the screen. Immunosuppressive sequences were employed for the ssNA because the toxicity of DNA donor templates from immune responses is one factor that may limit HDR efficiency. As shown in FIG. 1, modified donor templates designated D14 and D15 displayed robust HDR efficiency. Each donor employed an immunosuppressive sequence as described in WO2019094548A1.

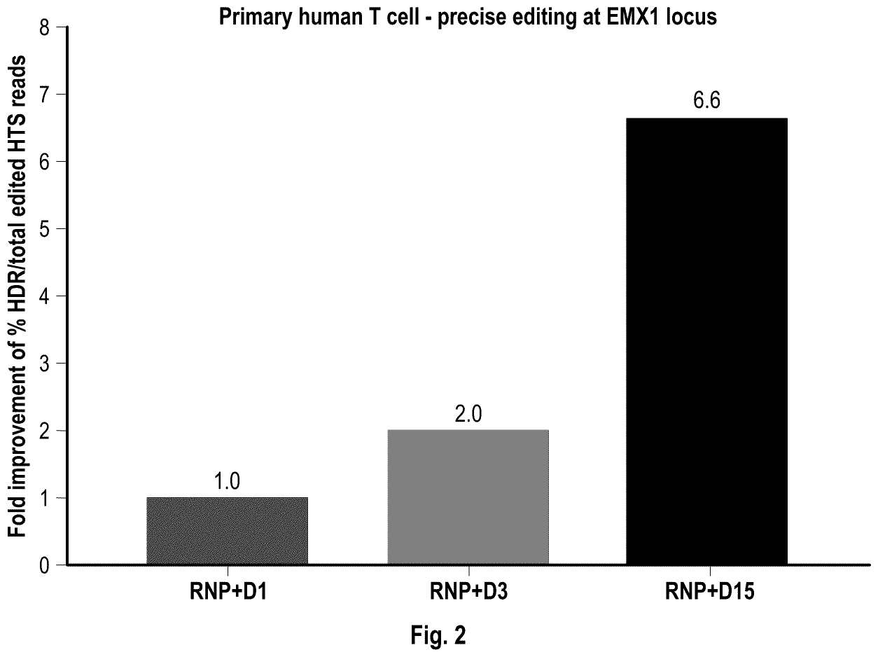

Jurkat cells are an immortalized line of human T lymphocyte cells. Given the surprisingly high HDR efficiency in these immune cells, select modified donors were next tested in primary human T cells. As shown in FIG. 2, the donor designated D15 had a 6.6-fold increase in HDR efficiency relative to an unmodified donor. The ability to efficiently integrate donor templates into primary immune cells is particularly useful for human therapeutics.

TABLE 1
Single stranded nucleic acids (ssNA) attached to the 5′ end of donor templates

| mod | Length | 2′ | | |
|---|---|---|---|---|
| sequence | (nt) | mods | PS | 5′-modification |
| S1 | 15 | OMe | 0 | (mG)(mG)(mA)(mA)(mG)(mG)(mG)(mC) |
| D3 | | | | (mC)(mG)(mA)(mG)(mC)(mG)(mC) |
| S1 | 15 | OMe | 3 | (mG)#(mG)#(mA)#(mA)(mG)(mG) |
| D4 | | | | (mG)(mC)(mC)(mG)(mA)(mG)(mC)(mG)(mC) |
| S1 | 15 | OMe | 8 | (mG)#(mG)#(mA)#(mA)#(mG)#(mG)# |
| D5 | | | | (mG)#(mC)#(mC)(mG)(mA)(mG)(mC)(mG)(mC) |
| S1 | 15 | OMe | 15 | (mG)#(mG)#(mA)#(mA)#(mG)#(mG)# |
| D6 | | | | (mG)#(mC)#(mC)#(mG)#(mA)#(mG)# |
| | | | | (mC)#(mG)#(mC)# |
| S1 | 15 | OMe | 3-3 | (mG)#(mG)#(mA)#(mA)(mG)(mG)(mG)(mC)(mC) |
| D7 | | | | (mG)(mA)(mG)(mC)(mG)(mC) - PEG - 3PS (5′ end |
| | | | | of donor template) |
| S1 | 15 | DNA | 0 | (dG)(dG)(dA)(dA)(dG)(dG)(dG)(dC) |
| D8 | | | | (dC)(dG)(dA)(dG)(dC)(dG)(dC) |
| S1 | 15 | DNA | 3 | (dG)#(dG)#(dA)#(dA)(dG)(dG) |
| D9 | | | | (dG)(dC)(dC)(dG)(dA)(dG)(dC)(dG)(dC) |
| S1 | 15 | DNA | 8 | (dG)#(dG)#(dA)#(dA)#(dG)#(dG)# |
| D10 | | | | (dG)#(dC)#(dC)(dG)(dA)(dG)(dC)(dG)(dC) |
| S1 | 15 | DNA | 15 | (dG)#(dG)#(dA)#(dA)#(dG)#(dG)# |
| D11 | | | | (dG)#(dC)#(dC)#(dG)#(dA)#(dG)# |
| | | | | (dC)#(dG)#(dC)# |
| AAG | 15 | OMe | 0 | (mA)(mA)(mG)(mA)(mA)(mG)(mA)(mA) |
| purine rich | | | | (mG)(mA)(mA)(mG)(mA)(mA)(mG) |
| D12 | | | | |
| TTC | 15 | OMe | 0 | (mU)(mU)(mC)(mU)(mU)(mC)(mU)(mU) |
| pyrimidine | | | | (mC)(mU)(mU)(mC)(mU)(mU)(mC) |
| rich | | | | |
| D13 | | | | |
| Immuno- | 24 | DNA | 24 | (dT)#(dT)#(dA)#(dG)#(dG)#(dG)#(dT)# |
| suppressive | | | | (dT)#(dA)#(dG)#(dG)#(dG)#(dT)#(dT)# |
| D14 | | | | (dA)#(dG)#(dG)#(dG)#(dT)#(dT)#(dA)# |
| | | | | (dG)#(dG)#(dG)# |
| Immuno- | 24 | OMe | 24 | (mU)#(mU)#(mA)#(mG)#(mG)#(mG)#(mU)# |
| suppressive | | | | (mU)#(mA)#(mG)#(mG)#(mG)#(mU)#(mU)# |
| D15 | | | | (mA)#(mG)#(mG)#(mG)#(mU)#(mU)#(mA)# |
| | | | | (mG)#(mG)#(mG)# |

In Table 1 above, “mN” corresponds to a nucleotide with a 2′-OMe modification; “dN” corresponds to a nucleotide with a 2′H modification; “#” corresponds to a phosphorothioate internucleotide linkage; “PEG” corresponds to poly ethylene glycol (tetra ethylene glycol for D7); and “3PS (5′ end of donor template)” corresponds to 3 consecutive phosphorothioate internucleotide linkages at the 5′ end of the donor template.

1. An isolated nucleic acid donor sequence comprising a 5′ end and a 3′ end, wherein a single-stranded nucleic acid (ssNA) moiety is attached at the isolated nucleic acid donor sequence 5′ end, and wherein the ssNA moiety comprises at least three phosphorothioate internucleotide linkages.

2. An isolated nucleic acid donor sequence comprising a 5′ end and a 3′ end, wherein a single-stranded RNA (ssNA) moiety is attached at the isolated nucleic acid donor sequence 5′ end, and wherein the ssNA moiety comprises an immunosuppressive sequence.

3. The isolated nucleic acid donor sequence of claim 1, wherein the isolated nucleic acid donor sequence comprises a region having portions of nucleic acid homology to a target sequence.

4. The isolated nucleic acid donor sequence of claim 1, wherein the isolated nucleic acid donor sequence is introduced into a target sequence by a homology-independent integration mechanism.

5. The isolated nucleic acid donor sequence of claim 4, wherein the homology-independent integration mechanism comprises engineering a cleavage site sequence into the isolated nucleic acid donor sequence, wherein the cleavage site sequence is also present in the target sequence.

6. The isolated nucleic acid donor sequence of claim 1, wherein the ssNA moiety comprises a single stranded RNA (ssRNA), optionally wherein the ssRNA comprises a sequence selected from the group consisting of:
(mG)(mG)(mA)(mA)(mG)(mG)(mG)(mC)(mC)(mG)(mA)(mG)(mC)(mG)(mC);
(mG) #(mG) #(mA) #(mA)(mG)(mG)(mG)(mC)(mC)(mG)(mA)(mG)(mC)(mG)(mC);
(mG) #(mG) #(mA) #(mA) #(mG) #(mG) #(mG) #(mC) #(mC)(mG)(mA)(mG)(mC)(mG)(mC);
(mG) #(mG) #(mA) #(mA) #(mG) #(mG) #(mG) #(mC) #(mC) #(mG) #(mA) #(mG) #(mC) #(mG) #(mC) #;
(dG)(dG)(dA)(dA)(dG)(dG)(dG)(dC)(dC)(dG)(dA)(dG)(dC)(dG)(dC);
(dG) #(dG) #(dA) #(dA)(dG)(dG)(dG)(dC)(dC)(dG)(dA)(dG)(dC)(dG)(dC);
(dG) #(dG) #(dA) #(dA) #(dG) #(dG) #(dG) #(dC) #(dC)(dG)(dA)(dG)(dC)(dG)(dC);
(dG) #(dG) #(dA) #(dA) #(dG) #(dG) #(dG) #(dC) #(dC) #(dG) #(dA) #(dG) #(dC) #(dG) #(dC) #;
(mA)(mA)(mG)(mA)(mA)(mG)(mA)(mA)(mG)(mA)(mA)(mG)(mA)(mA)(mG);
(mU)(mU)(mC)(mU)(mU)(mC)(mU)(mU)(mC)(mU)(mU)(mC)(mU)(mU)(mC);
(dT) #(dT) #(dA) #(dG) #(dG) #(dG) #(dT) #(dT) #(dA) #(dG) #(dG) #(dG) #(dT) #(dT) #(dA) #(dG) #(dG) #(dG) #(dT) #(dT) #(dA) #(dG) #(dG) #(dG) #; or
(mU) #(mU) #(mA) #(mG) #(mG) #(mG) #(mU) #(mU) #(mA) #(mG) #(mG) #(mG) #(mU) #(mU) #(mA) #(mG) #(mG) #(mG) #(mU) #(mU) #(mA) #(mG) #(mG) #(mG) #,
wherein:
“m” corresponds to a nucleotide with a 2′-OMe modification;
“d” corresponds to a nucleotide with a 2′H modification; and
“#” corresponds to a phosphorothioate internucleotide linkage.

7. The isolated nucleic acid donor sequence of claim 1, wherein the ssNA moiety is attached to the 5′ end of the isolated nucleic acid donor sequence with a linker.

8. The isolated nucleic acid donor sequence of claim 7, wherein the linker is selected from the group consisting of aminoethoxyethoxyacetate (AEEA), aminohexanoic acid, oligoglycine, ethylene glycol, polyethylene glycol (PEG), amino C6, and amino C12.

9. (canceled)

10. The isolated nucleic acid donor sequence of claim 1, wherein the ssNA moiety further comprises one or more of ethylene glycol, polyethylene glycol (PEG), a polyamine having at least two amino groups, and an alkanediol attached to the 5′ end of the ssNA moiety.

11. The isolated nucleic acid donor sequence of claim 1, wherein the ssNA moiety comprises one or more modified nucleotides.

12. The isolated nucleic acid donor sequence of claim 11, wherein the one or more modified nucleotides are selected from the group consisting of a 2′-O-alkyl modified nucleotide, a 2′-fluoro modified nucleotide, a nucleotide comprising a 5′-phosphorothioate group, a 2′-deoxy-modified nucleotide, a locked nucleic acid (LNA), a bridged nucleotide, a constrained nucleotide, a bicyclic nucleotide, an abasic nucleotide, a 2′-amino-modified nucleotide, a 2′-alkyl-modified nucleotide, a morpholino nucleotide, a phosphoramidate, a peptide nucleic acid, and a non-natural base comprising nucleotide.

13. (canceled)

14. The isolated nucleic acid donor sequence of claim 1, wherein the ssNA moiety comprises one or more modified internucleotide linkages.

15. The isolated nucleic acid donor sequence of claim 14, wherein the one or more modified internucleotide linkages comprise a phosphorothioate internucleotide linkage.

16. The isolated nucleic acid donor sequence of claim 14, wherein the one or more modified internucleotide linkages comprise a modified internucleotide linkage of Formula I: 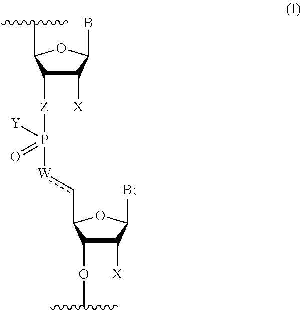
wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O″, OH, OR, NH, NH₂, S, and SH,
wherein R is a protecting group;
Z is selected from the group consisting of O and CH₂; [symbol]
is an optional double bond.

17. (canceled)

18. The nucleic acid donor sequence of claim 1, wherein the ssNA moiety is about 8 bases in length to about 30 bases in length.

19-46. (canceled)

47. The isolated nucleic acid donor sequence of claim 1, wherein the isolated nucleic acid donor sequence enhances donor genome integration in an immune cell or a hematopoietic stem or progenitor cell (HSPC) relative to a nucleic acid donor sequence lacking the ssNA moiety.

48-50. (canceled)

51. An isolated nucleic acid donor sequence comprising a 5′ end and a 3′ end, wherein the isolated nucleic acid donor sequence comprises one or more phosphorothioate internucleotide linkages at the 5′ end.

52-100. (canceled)

101. A method of introducing a nucleic acid donor sequence into a target sequence of a genome in a cell, the method comprising:
i) contacting the cell with the isolated nucleic acid donor sequence of claim 1; and
ii) contacting the cell with an agent that creates a double-stranded break at or near the target sequence.

102-104. (canceled)

105. A genome-editing system comprising:
i) the isolated nucleic acid donor sequence of claim 1; and
ii) an agent that creates a double-stranded break at or near a target sequence.

106. (canceled)

107. A method of introducing a nucleic acid donor sequence into a target sequence of a genome in an immune cell or a hematopoietic stem or progenitor cell (HSPC), the method comprising:
i) contacting the cell with a nucleic acid donor sequence comprising a single-stranded nucleic acid (ssNA) moiety; and
ii) contacting the cell with an agent that creates a double-stranded break at or near the target sequence.

108-111. (canceled)


SEQUENCE LISTING (38 sequences)

SEQ ID NO: 1 - RNA, 15 nt, synthetic construct
  source 1..15: mol_type = other RNA
  modified_base order(1..2,5..7,10,12,14): mod_base = OTHER; note = 2-prime-OMe-guanosine
  modified_base order(3..4,11): mod_base = OTHER; note = 2-prime-OMe-adenosine
  modified_base order(8..9,13,15): mod_base = OTHER; note = 2-prime-OMe-cytidine
ggaagggccgagcgc

SEQ ID NO: 2 - RNA, 15 nt, synthetic construct
  source 1..15: mol_type = other RNA
  modified_base order(1..2,5..7,10,12,14): mod_base = OTHER; note = 2-prime-OMe-guanosine
  modified_base order(3..4,11): mod_base = OTHER; note = 2-prime-OMe-adenosine
  modified_base order(8..9,13,15): mod_base = OTHER; note = 2-prime-OMe-cytidine
  misc_binding order(1..2,2..3,3..4): bound_moiety = phosphorothioate
ggaagggccgagcgc

SEQ ID NO: 3 - RNA, 15 nt, synthetic construct
  source 1..15: mol_type = other RNA
  modified_base order(1..2,5..7,10,12,14): mod_base = OTHER; note = 2-prime-OMe-guanosine
  modified_base order(3..4,11): mod_base = OTHER; note = 2-prime-OMe-adenosine
  modified_base order(8..9,13,15): mod_base = OTHER; note = 2-prime-OMe-cytidine
  misc_binding order(1..2,2..3,3..4,4..5,5..6,6..7,7..8,8..9): bound_moiety = phosphorothioate
ggaagggccgagcgc

SEQ ID NO: 4 - RNA, 15 nt, synthetic construct
  source 1..15: mol_type = other RNA
  modified_base order(1..2,5..7,10,12,14): mod_base = OTHER; note = 2-prime-OMe-guanosine
  modified_base order(3..4,11): mod_base = OTHER; note = 2-prime-OMe-adenosine
  modified_base order(8..9,13,15): mod_base = OTHER; note = 2-prime-OMe-cytidine
  misc_binding order(1..2,2..3,3..4,4..5,5..6,6..7,7..8,8..9,9..10,10..11,11..12,12..13,13..14,14..15,15): bound_moiety = phosphorothioate
ggaagggccgagcgc

SEQ ID NO: 5 - RNA, 15 nt, synthetic construct
  source 1..15: mol_type = other RNA
  modified_base order(1..2,5..7,10,12,14): mod_base = OTHER; note = 2-prime-OMe-guanosine
  modified_base order(3..4,11): mod_base = OTHER; note = 2-prime-OMe-adenosine
  modified_base order(8..9,13,15): mod_base = OTHER; note = 2-prime-OMe-cytidine
  misc_binding order(1..2,2..3,3..4): bound_moiety = phosphorothioate
ggaagggccgagcgc

SEQ ID NO: 6 - RNA, 15 nt, synthetic construct
  source 1..15: mol_type = other RNA
  modified_base order(1..2,5..7,10,12,14): mod_base = OTHER; note = 2-prime-H- guanosine
  modified_base order(3..4,11): mod_base = OTHER; note = 2-prime-H- adenosine
  modified_base order(8..9,13,15): mod_base = OTHER; note = 2-prime-H-cytidine
ggaagggccgagcgc

SEQ ID NO: 7 - RNA, 15 nt, synthetic construct
  source 1..15: mol_type = other RNA
  modified_base order(1..2,5..7,10,12,14): mod_base = OTHER; note = 2-prime-H- guanosine
  modified_base order(3..4,11): mod_base = OTHER; note = 2-prime-H- adenosine
  modified_base order(8..9,13,15): mod_base = OTHER; note = 2-prime-H-cytidine
  misc_binding order(1..2,2..3,3..4): bound_moiety = phosphorothioate
ggaagggccgagcgc

SEQ ID NO: 8 - RNA, 15 nt, synthetic construct
  source 1..15: mol_type = other RNA
  modified_base order(1..2,5..7,10,12,14): mod_base = OTHER; note = 2-prime-H- guanosine
  modified_base order(3..4,11): mod_base = OTHER; note = 2-prime-H- adenosine
  modified_base order(8..9,13,15): mod_base = OTHER; note = 2-prime-H-cytidine
  misc_binding order(1..2,2..3,3..4,4..5,5..6,6..7,7..8,8..9): bound_moiety = phosphorothioate
ggaagggccgagcgc

SEQ ID NO: 9 - RNA, 15 nt, synthetic construct
  source 1..15: mol_type = other RNA
  modified_base order(1..2,5..7,10,12,14): mod_base = OTHER; note = 2-prime-H- guanosine
  modified_base order(3..4,11): mod_base = OTHER; note = 2-prime-H- adenosine
  modified_base order(8..9,13,15): mod_base = OTHER; note = 2-prime-H-cytidine
  misc_binding order(1..2,2..3,3..4,4..5,5..6,6..7,7..8,8..9,9..10,10..11,11..12,12..13,13..14,14..15,15): bound_moiety = phosphorothioate
ggaagggccgagcgc

SEQ ID NO: 10 - RNA, 15 nt, synthetic construct
  source 1..15: mol_type = other RNA
  modified_base order(1..2,4..5,7..8,10..11,13..14): mod_base = OTHER; note = 2-prime-OMe-adenosine
  modified_base order(3,6,9,12,15): mod_base = OTHER; note = 2-prime-OMe-guanosine
aagaagaagaagaag

SEQ ID NO: 11 - RNA, 15 nt, synthetic construct
  source 1..15: mol_type = other RNA
  modified_base order(1..2,4..5,7..8,10..11,13..14): mod_base = OTHER; note = 2-prime-OMe-uridine
  modified_base order(3,6,9,12,15): mod_base = OTHER; note = 2-prime-OMe-cytidine
ttcttcttcttcttc

SEQ ID NO: 12 - RNA, 24 nt, synthetic construct
  source 1..24: mol_type = other RNA
  modified_base order(1..2,7..8,13..14,19..20): mod_base = OTHER; note = 2-prime-H-thymine
  modified_base order(3,9,15,21): mod_base = OTHER; note = 2-prime-H- adenosine
  modified_base order(4..6,10..12,16..18,22..24): mod_base = OTHER; note = 2-prime-H- guanosine
  misc_binding order(1..2,2..3,3..4,4..5,5..6,6..7,7..8,8..9,9..10,10..11,11..12,12..13,13..14,14..15,15..16,16..17,17..18,18..19,19..20,20..21,21..22,22..23,23..24,24): bound_moiety = phosphorothioate
ttagggttagggttagggttaggg

SEQ ID NO: 13 - RNA, 24 nt, synthetic construct
  source 1..24: mol_type = other RNA
  modified_base order(1..2,7..8,13..14,19..20): mod_base = OTHER; note = 2-prime-OMe-uridine
  modified_base order(3,9,15,21): mod_base = OTHER; note = 2-prime-OMe-adenosine
  modified_base order(4..6,10..12,16..18,22..24): mod_base = OTHER; note = 2-prime-OMe-guanosine
  misc_binding order(1..2,2..3,3..4,4..5,5..6,6..7,7..8,8..9,9..10,10..11,11..12,12..13,13..14,14..15,15..16,16..17,17..18,18..19,19..20,20..21,21..22,22..23,23..24,24): bound_moiety = phosphorothioate
ttagggttagggttagggttaggg

SEQ ID NO: 14 - RNA, 24 nt, synthetic construct
  source 1..24: mol_type = other RNA
  modified_base order(1..2,7..8,13..14,19..20): mod_base = OTHER; note = 2-prime-H-uridine
  modified_base order(3,9,15,21): mod_base = OTHER; note = 2-prime-H- adenosine
  modified_base order(4..6,10..12,16..18,22..24): mod_base = OTHER; note = 2-prime-H- guanosine
  misc_binding order(1..2,2..3,3..4,4..5,5..6,6..7,7..8,8..9,9..10,10..11,11..12,12..13,13..14,14..15,15..16,16..17,17..18,18..19,19..20,20..21,21..22,22..23,23..24,24): bound_moiety = phosphorothioate
ttagggttagggttagggttaggg

SEQ ID NO: 15 - protein, 6 aa, synthetic construct
PKKKRK

SEQ ID NO: 16 - RNA, 12 nt, synthetic construct
  source 1..12: mol_type = other RNA
ttagggttaggg

SEQ ID NO: 17 - RNA, 18 nt, synthetic construct
  source 1..18: mol_type = other RNA
ttagggttagggttaggg

SEQ ID NO: 18 - RNA, 24 nt, synthetic construct
  source 1..24: mol_type = other RNA
ttagggttagggttagggttaggg

SEQ ID NO: 19 - RNA, 30 nt, synthetic construct
  source 1..30: mol_type = other RNA
ttagggttagggttagggttagggttaggg

SEQ ID NO: 20 - DNA, 12 bp, synthetic construct
  source 1..12: mol_type = other DNA
ttagggttaggg

SEQ ID NO: 21 - DNA, 18 bp, synthetic construct
  source 1..18: mol_type = other DNA
ttagggttagggttaggg

SEQ ID NO: 22 - DNA, 24 bp, synthetic construct
  source 1..24: mol_type = other DNA
ttagggttagggttagggttaggg

SEQ ID NO: 23 - DNA, 30 bp, synthetic construct
  source 1..30: mol_type = other DNA
ttagggttagggttagggttagggttaggg

SEQ ID NO: 24 - DNA, 15 bp, synthetic construct
  source 1..15: mol_type = other DNA
tcctggaggggttgt

SEQ ID NO: 25 - DNA, 20 bp, synthetic construct
  source 1..20: mol_type = other DNA
tgcttgcaagcttgcaagca

SEQ ID NO: 26 - DNA, 24 bp, synthetic construct
  source 1..24: mol_type = other DNA
ttcccatccaggcctggatgggaa

SEQ ID NO: 27 - DNA, 24 bp, synthetic construct
  source 1..24: mol_type = other DNA
cctggatgggaacttaccgctgca

SEQ ID NO: 28 - DNA, 24 bp, synthetic construct
  source 1..24: mol_type = other DNA
cttaccgctgcacctggatgggaa

SEQ ID NO: 29 - DNA, 24 bp, synthetic construct
  source 1..24: mol_type = other DNA
cctggatgggaattcccatccagg

SEQ ID NO: 30 - DNA, 22 bp, synthetic construct
  source 1..22: mol_type = other DNA
tgactgtgaaggttagagatga

SEQ ID NO: 31 - DNA, 21 bp, synthetic construct
  source 1..21: mol_type = other DNA
ctcctattgggggtttcctat

SEQ ID NO: 32 - DNA, 20 bp, synthetic construct
  source 1..20: mol_type = other DNA
tggcgcgcacccacggcctg

SEQ ID NO: 33 - DNA, 12 bp, synthetic construct
  source 1..12: mol_type = other DNA
cctggatgggaa

SEQ ID NO: 34 - DNA, 10 bp, synthetic construct
  source 1..10: mol_type = other DNA
cctggcgggg

SEQ ID NO: 35 - DNA, 15 bp, synthetic construct
  source 1..15: mol_type = other DNA
tcctggatgggaagt

SEQ ID NO: 36 - DNA, 15 bp, synthetic construct
  source 1..15: mol_type = other DNA
tcctggaggggaagt

SEQ ID NO: 37 - DNA, 15 bp, synthetic construct
  source 1..15: mol_type = other DNA
tcctggcggggaagt

SEQ ID NO: 38 - protein, 6 aa, synthetic construct
NNGRRT